(19) United States
(12) Patent Application Publication
(10) Pub. No.: US 2014/0024123 A1
(43) Pub. Date: Jan. 23, 2014

(54) Aqueous Solution Containing Partial Ras Polypeptide and Method for Screening Inhibitor of Ras Function

(71) Applicants: Tohru Kataoka, Kobe-shi (JP); Fumi Shima, Kobe-shi (JP); Atsuo Tamuta, Kobe-shi (JP); Mitsugu Araki, Kobe-shi (JP)

(72) Inventors: Tohru Kataoka, Kobe-shi (JP); Fumi Shima, Kobe-shi (JP); Atsuo Tamuta, Kobe-shi (JP); Mitsugu Araki, Kobe-shi (JP)

(73) Assignee: NATIONAL UNIVERSITY CORPORATION KOBE UNIVERSITY, Kobe-shi, Hyogo (JP)

(21) Appl. No.: 13/984,027

(22) PCT Filed: Jan. 31, 2012
(86) PCT No.: PCT/JP2012/052078

§ 371 Date: Oct. 8, 2013

(30) Foreign Application Priority Data
Feb. 7, 2011 (JP) 2011-023695

(51) Int. Cl. C07K 14/47; C07C 337/02

(52) U.S. Cl. CPC C07K 14/4722; C07C 337/02
USPC 436/86; 564/18; 530/408

(57) ABSTRACT

Provided is a screening method for a more effective Ras inhibitor. Further provided are amino acid residues at a site important for an interaction with a Ras inhibitor in a Ras polypeptide. By confirming a difference between structural information on the Ras polypeptide by NMR and structural information between a complex of the Ras polypeptide and a seed compound by NMR, a more effective lead compound than the seed compound can be selected. As a result of analysis based on the structural information on the Ras polypeptide by NMR, the amino acid residues capable of interacting with a Ras inhibitor candidate (a) are K5, E37, D38, S39, L56, E63, Y64, A66, M67, Q70, Y71, R73, and T74 with reference to an amino acid sequence set forth in SEQ ID NO: 1.

### TECHNICAL FIELD

The present invention relates to a screening method for selection of a more effective Ras inhibitor, including using nuclear magnetic resonance method (NMR) information. More specifically, the present invention relates to a screening method for a Ras inhibitor, including utilizing structural information about a Ras polypeptide that adopts a conformation having a pocket on a molecular surface of Ras by NMR.

The present application claims priority of Japanese Patent Application No. 2011-23695, which is incorporated herein by reference.

### BACKGROUND ART

Ras, a product of a ras proto-oncogene, is a low molecular weight G protein. Ras has three isoforms, i.e., H-Ras, N-Ras, and K-Ras in mammals. Ras also has homologs, which exhibit similar amino acid sequences, such as M-Ras and Rap. Members including those isoforms and homologs form the Ras family. In human cancers, constitutive activation of a Ras function through a mutation of any one of H-Ras, N-Ras, and K-Ras is observed at a high frequency. Hence, Ras is considered as a promising molecular target for development of anti-cancer drugs.

Ras regulates signaling while cycling between Ras in a GTP-bound form as an activated form (Ras-GTP) and Ras in a GDP-bound form as an inactivated form (Ras-GDP). When a ligand (extracellular signal) such as a cell growth factor binds to a cell membrane receptor, Ras-GDP is activated through the conversion of GDP included therein to GTP, and Ras-GTP binds to a target molecule to induce cell growth signaling. After that, Ras-GTP returns to Ras-GDP through the action of a factor for promoting a GTP-hydrolyzing (GTPase) activity inherent to Ras (GTPase-activating protein: GAP), and is present in an inactivated state until the next signal arrives. In human cancers, it is suggested that mutations (mutation in an activated form) of Ras prevents its intrinsic and GAP-mediated GTP-hydrolyzing activity, and hence a percentage of Ras in a GTP-bound form in cells is increased, resulting in sustained cell growth signaling leading to cancer development.

In addition, it has hitherto been pointed out that Ras-GTP, a GTP-bound form, exists in two interconverting conformations (state 1 and state 2) based on ³¹P-nuclear magnetic resonance (NMR). State 2 is a bona fide activated form capable of binding to a target protein to induce signaling, whereas state 1 is an inactivated form incapable of binding to a target protein. The conformation of state 2 has been elucidated by X-ray crystallography and NMR analysis.

Elucidation of the conformation of state 1 of H-Ras-GTP has been attempted, but the conformation has not yet been elucidated completely. H-Ras T35S (amino acid residues 1 to 189), which is a H-Ras mutant, was subjected to X-ray crystallography (Non Patent Literature 1). However, it is concluded that the state 1 structure disclosed in Non Patent Literature 1 is not a complete one because an electron density for the main chains of a plurality of amino acid residues was completely missing. Further, such electron density-missing residues were located in the two switch regions (switches I and II) which plays crucial roles for recognition of effectors and regulators such as GEFs and GAPS.

Focused on M-Ras, a member of the Ras family, state 1 of wild-type M-Ras-GTP was subjected to X-ray crystallographic analysis (PDB ID: 1X1S) (Non Patent Literature 2). The results revealed that state 1 of M-Ras-GTP had a pocket, which was not observed in the state 2 structure of Ras-GTP or that of Ras-GDP and such pocket was surrounded by the switch I and switch II regions. However, the state 1 structure of M-Ras-GTP disclosed in Non Patent Literature 2 was incomplete because of the partial deletion of the electron density for the residues surrounding the pocket.

Two kinds of conformations (forms 1 and 2) of state 1 were determined as a result of X-ray crystallographic analysis of H-Ras T35S (amino acid residues 1 to 166), which is a H-Ras mutant, under different conditions from those of Non Patent Literature 1 (Non Patent Literature 3). It was concluded that form 2 is not a complete conformation because the electron density for the main chains of a plurality of amino acid residues in switch II was missing, whereas form 1 showed a complete state 1 structure whose electron density for all of the amino acid residues was completely visible. However, even for form 1 as well as other incomplete state 1 structures, the information on the critical amino acid residues which form the specific drug-binding sites remained unknown.

Ras inhibitor is a promising candidate for anti-cancer drugs. Hence, there is a strong demand for such amino acid residue information for a binding region of a medicament of the state 1 structure of Ras-GTP as to be able to be used for computer docking simulation for the development of the Ras inhibitor. There is also a strong demand for structural information for constructing a most suitable lead compound based on a seed compound having a Ras inhibition action.

### CITATION LIST

### Non Patent Literature

[NPL 1] Spoerner, M., et al., Proc Natl Acad Sci USA. 2001 Apr. 24; 98(9): 4944-9.
[NPL 2] Ye M., et al., J Biol Chem. 2005 Sep. 2; 280(35): 31267-75.
[NPL 3] Shima F., et al., J Biol Chem. 2010 May 17; 285(29): 22696-705.

### SUMMARY OF INVENTION

### Technical Problem

An object of the present invention is to provide a screening method for a Ras inhibitor. Another object of the present invention is to provide amino acid residues at a site important for an interaction with a Ras inhibitor in a Ras polypeptide.

### Solution to Problem

In order to achieve the objects, the inventors of the present invention have made intensive studies, and have made analysis based on the structural information on a Ras polypeptide by NMR and that on a complex between a seed compound exhibiting efficacy as a Ras inhibitor and a Ras polypeptide by NMR. As a result, the inventors have been able to confirm the site important for the interaction between the Ras polypeptide and the Ras inhibitor. Thus, the present invention has been completed. The inventors have also found that a more effective lead compound than the seed compound can be selected by confirming a difference between the structural information on the Ras polypeptide by NMR and that on the complex between the Ras polypeptide and the seed compound by NMR. Thus, a screening method for a Ras inhibitor according to the present invention has been completed.

That is, the present invention includes the following items.

1. A screening method for a Ras inhibitor (A), including the following steps of:
1) obtaining an NMR signal from a Ras partial polypeptide formed of an amino acid sequence set forth in SEQ ID NO: 1 or an amino acid sequence having substitutions, deletions, or additions of one to three amino acids in the amino acid sequence set forth in SEQ ID NO: 1;
2) obtaining an NMR signal from a complex obtained by bringing a Ras inhibitor candidate (a) into contact with the Ras partial polypeptide as defined in the step 1); and
3) selecting a Ras inhibitor (A) as a derivative of the Ras inhibitor candidate (a) by analyzing a difference between the NMR signal obtained in the step 1) and the NMR signal obtained in the step 2).
2. A screening method for a Ras inhibitor candidate (a), including selecting a substance capable of interacting with at least three or more amino acid residues selected from, with reference to an amino acid sequence set forth in SEQ ID NO: 1, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in a Ras partial polypeptide formed of the amino acid sequence set forth in SEQ ID NO: 1 or an amino acid sequence having substitutions, deletions, or additions of one to three amino acids in the amino acid sequence set forth in SEQ ID NO: 1.
3. A screening method for a Ras inhibitor candidate (a) according to the above-mentioned item 2, further including the following steps of:
1) producing a polypeptide in which the at least the three or more amino acid residues selected from lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in the Ras partial polypeptide as defined in the above-mentioned item 2 are each labeled each with an isotope measurable by NMR to obtain an NMR signal from the labeled polypeptide;
2) bringing a candidate into contact with the labeled polypeptide produced in the step 1) to obtain an NMR signal from a complex between the candidate and the labeled polypeptide; and
3) selecting, as the Ras inhibitor candidate (a), a substance capable of interacting with the labeled polypeptide based on a difference between the NMR signal obtained in the step 1) and the NMR signal obtained in the step 2).
4. A screening method for a Ras inhibitor (A) according to the above-mentioned item 1 or a Ras inhibitor candidate (a) according to the above-mentioned item 2 or 3, in which the Ras partial polypeptide includes a polypeptide formed of an amino acid sequence in which threonine at position 35 is substituted by serine in the amino acid sequence set forth in SEQ ID NO: 1.
5. A screening method for a Ras inhibitor (A) according to the above-mentioned item 1, in which the Ras inhibitor candidate (a) is represented by the following general formula (I):

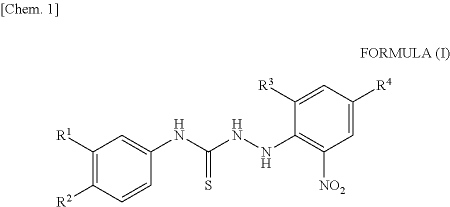

where R¹, R², R³, and R⁴ each independently represent an atom or group selected from the group consisting of a hydrogen atom, a halogen group, a lower alkyl group, a nitro group, and a trifluoromethyl group.
6. A Ras inhibitor, including a Ras inhibitor candidate (a) as an active ingredient, represented by the following general formula (I):

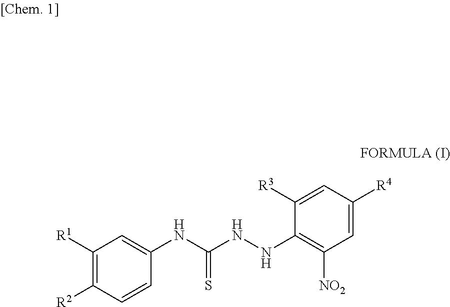

where R¹, R², R³, and R⁴ each independently represent an atom or group selected from the group consisting of a hydrogen atom, a halogen group, a lower alkyl group, a nitro group, and a trifluoromethyl group.
7. A complex between a Ras partial polypeptide formed of an amino acid sequence set forth in SEQ ID NO: 1 or an amino acid sequence having substitutions, deletions, or additions of one to three amino acids in the amino acid sequence set forth in SEQ ID NO: 1, and a Ras inhibitor candidate (a) represented by the following general formula (I):

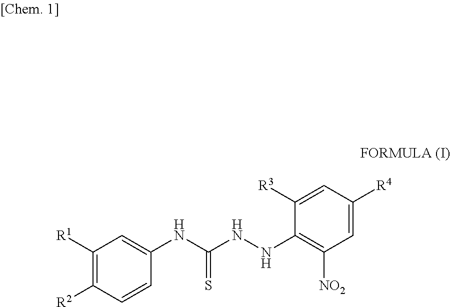

where R¹, R², R³, and R⁴ each independently represent an atom or group selected from the group consisting of a hydrogen atom, a halogen group, a lower alkyl group, a nitro group, and a trifluoromethyl group.
8. A complex according to the above-mentioned item 7, in which the Ras partial polypeptide includes a polypeptide formed of an amino acid sequence in which threonine at position 35 is substituted by serine in the amino acid sequence set forth in SEQ ID NO: 1.
9. A complex according to the above-mentioned item 7 or 8, in which the complex is specified by an NMR signal.
10. A Ras partial polypeptide-containing aqueous solution, including a Ras partial polypeptide formed of an amino acid sequence set forth in SEQ ID NO: 1 or an amino acid sequence having substitutions, deletions, or additions of one to three amino acids in the amino acid sequence set forth in SEQ ID NO: 1, in which a structure of the Ras partial polypeptide is as defined in the following items 1) and 2):
1) the structure of the Ras partial polypeptide includes a conformation having a pocket capable of binding to a specified substance; and
2) the specified substance includes a substance capable of interacting with at least three or more amino acid residues selected from, with reference to the amino acid sequence set forth in SEQ ID NO: 1, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in the Ras partial polypeptide.
11. A Ras partial polypeptide-containing aqueous solution according to the above-mentioned item 10, in which the Ras partial polypeptide includes a polypeptide formed of an amino acid sequence in which threonine at position 35 is substituted by serine in the amino acid sequence set forth in SEQ ID NO: 1.
12. A Ras partial polypeptide-containing aqueous solution according to the above-mentioned item 10 or 11, in which the specified substance includes a Ras inhibitor (a) represented by the general formula (I).

### Advantageous Effects of Invention

In the present invention, it was possible to provide the amino acid residues at the site important for the interaction with the Ras inhibitor in the Ras polypeptide. In addition, through the confirmation of the difference between the structural information on the Ras polypeptide by NMR and the structural information on the complex between the Ras polypeptide and the seed compound by NMR, a novel derivative can be designed and synthesized in such a form that a substituent at a high risk of expressing toxicity or a metabolically unstable chemical structure is substituted by its biological equivalent, and a more effective lead compound than the seed compound can be selected. According to such screening method, a lead compound having a more effective substituent can be constructed as the Ras inhibitor based on a certain seed compound.

### BRIEF DESCRIPTION OF DRAWINGS

FIG. 1 A conceptual diagram showing a structural model of a Ras partial polypeptide obtained by construction based an NMR signal, the diagram also including a structural fluctuation associated with a protein function.

FIG. 2 A conceptual diagram schematically showing a site of a pocket estimated from a structural model generated by using obtained NMR signal, the diagram also including a structural fluctuation related to a protein function.

FIG. 3 A conceptual diagram showing a structural model of a complex between a Ras inhibitor candidate (a) and a Ras partial polypeptide generated by using the obtained NMR signal, the diagram also including a structural fluctuation related to a protein function.

FIG. 4 A graph showing biochemical activity verification test results of Compound (II), Compound (III), and Compound (IV) (Experimental Example 1).

FIG. 5 A graph showing cellular activities of Compound (II), Compound (III), and Compound (IV) (Experimental Example 2).

FIG. 6 NMR spectra of a complex between a Ras partial polypeptide and Compound (III) (Example 2).

FIG. 7 An image showing amino acid residues capable of interacting with Compound (III) in a Ras partial polypeptide (Example 4).

### DESCRIPTION OF EMBODIMENTS

The present invention relates to a screening method for selection of an even more effective Ras inhibitor from seed compounds, including using NMR information. More specifically, the present invention relates to a screening method for a Ras inhibitor, including utilizing structural information on a Ras polypeptide by NMR, that adopts a conformation having a pocket on a molecular surface of Ras.

In the present invention, the “candidate” may be any of known and novel ones. Further, structures, origins, physical properties, and the like thereof are not particularly limited. In addition, the candidate may be any of a natural compound, a synthetic compound, a high molecular weight compound, a low molecular weight compound, a peptide, and a nucleic acid analog. A known program has only to be used for the conversion of the structure of the candidate into coordinates. For example, as a program that converts the structure of the low molecular weight compound into coordinates, CORINA (http://www2.chemie.uni-erlangen.de/software/corina/index.html), Concord (http://www.tripos.com/sciTech/inSilicoDisc/chemInfo/concord.html), Converter, COFIMA (http://www.bpc.uni-frankfurt.de/guentert/wiki/index.php/Software), or the like may be utilized. CYANA (http://www.las.jp/products/s08_cyana/index.html) or CNS (http://cns.csb.yale.edu/v1.1/) may be utilized for a peptide and a nucleic acid.

In the present invention, the “Ras inhibitor candidate (a)” refers to a substance having an inhibitory activity on Ras selected from the above-mentioned candidates by primary screening, that is, a seed compound having an inhibitory activity on Ras function. Further, the “Ras inhibitor (A)” as used herein means a derivative selected based on the information on the Ras inhibitor candidate (a), that is, a lead compound additionally optimized from the Ras inhibitor candidate (a).

(Screening Method for Ras Inhibitor (A))

As an aspect of a screening method for a Ras inhibitor (A) of the present invention, there is given a screening method including the following steps of:

1) obtaining an NMR signal from a Ras partial polypeptide;
2) obtaining an NMR signal from a complex obtained by bringing a Ras inhibitor candidate (a) into contact with the Ras partial polypeptide as defined in the step 1); and
3) selecting a derivative of the Ras inhibitor candidate (a) by analyzing a difference between the NMR signal obtained in the step 1) and the NMR signal obtained in the step 2).

A Ras partial polypeptide of the present invention is suitably a Ras mutant partial polypeptide obtained by introducing a mutation leading to the formation of a conformation having a pocket on the molecular surface into a partial polypeptide of a Ras protein. In this connection, the Ras partial polypeptide needs to be a Ras partial polypeptide bound to GTP or a GTP analog, that is, a Ras partial polypeptide in a GTP-bound form in order that the polypeptide forms a conformation having a pocket on the molecular surface. The GTP analog includes one having a GTP-like backbone subjected to chemical modification or salt formation, and is capable of binding to a GTP-binding site. Thus, in the present invention, it is meant that crystals, X-ray crystallographic analysis results, and NMR signals to be obtained for the Ras partial polypeptide are all obtained from a Ras partial polypeptide in a GTP-bound form (hereinafter sometimes referred to as “Ras-GTP”) or a complex between a Ras inhibitor candidate (a) and Ras-GTP. It is known that the pocket on the molecular surface of Ras is not present on the molecular surface of a Ras partial polypeptide in a GDP-bound form (hereinafter sometimes referred to as “Ras-GDP”), and is present in Ras-GTP. The pocket is surrounded by the regions called the switch regions (including the switch I region and the switch II region) in an amino acid sequence of Ras. The switch regions are defined as the two regions that undergo drastic conformational changes between the Ras partial polypeptide in a GDP-bound form and the Ras partial polypeptide in a GTP-bound form and are the regions important for Ras to recognize and activate a target molecule, and GTP binds to the vicinity of the regions.

The Ras partial polypeptide of the present invention constituting Ras-GTP specifically refers to a polypeptide formed of the following amino acid sequence set forth in SEQ ID NO: 1 (at positions 1 to 166 of H-Ras) or a H-Ras mutant partial polypeptide formed of an amino acid sequence having substitutions, deletions, or additions of one to three amino acids in the amino acid sequence set forth in SEQ ID NO: 1.

SEQ ID NO: 1:
H-Ras (amino acid residues 1 to 166):
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHQYRE
QIKRVKDSDDVPMVLVGNKCDLAARTVESRQAQDLARSYGIPYIETSAK
TRQGVEDAFYTLVREIRQH

The Ras partial polypeptide formed of an amino acid sequence having substitutions, deletions, or additions of one to three amino acids in the amino acid sequence set forth in SEQ ID NO: 1 refers to a polypeptide having substitutions, deletions, or additions of one to three amino acids in a region formed of amino acids at positions 27 to 43, preferably a region formed of amino acids at positions 30 to 41 in the amino acid sequence set forth in SEQ ID NO: 1. The mutation is preferably (a) substitution(s) of (an) amino acid(s) at position(s) 31 and/or 35, particularly preferably a substitution of threonine at position 35. The threonine at position 35 is preferably substituted by serine. A H-Ras mutant polypeptide in which the threonine at position 35 is substituted by serine is hereinafter referred to as “H-Ras T35S.” As used herein, the Ras partial polypeptide of the present invention is meant to encompass H-Ras T35S as well.

An amino acid mutation in a polypeptide may be introduced by a site-directed mutagenesis method well known to a person skilled in the art, and may be introduced using a kit (e.g., QuikChange™Site-Directed Mutagenesis Kit (STRATAGENE)). Alternatively, a DNA fragment encoding an amino acid mutation may be introduced or substituted using a genetic engineering technique well known to a person skilled in the art. For example, a Ras mutant polypeptide may be produced by using a PCR reaction, a restriction enzyme reaction, a ligation reaction, and the like in combination. Specifically, for example, H-Ras T35S may be produced by amplifying a DNA fragment corresponding to amino acid residues 1 to 166 by ordinary PCR using, as a template, H-Ras T35S (pBR322H-Ras T35S (amino acid residues 1 to 189)) subcloned into a vector pBR322, and cloning the amplified fragment into pGEX-6p-1.

A Ras inhibitor candidate (a) to be used in the screening method for a Ras inhibitor (A) of the present invention may be selected from candidates by the following method.

The method includes selecting, from candidates, a substance capable of interacting with at least three or more amino acid residues selected from, with reference to the amino acid sequence set forth in SEQ ID NO: 1 in the Ras partial polypeptide of the present invention, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in the amino acid sequence set forth in SEQ ID NO: 1. The method preferably includes selecting, from candidates, a substance capable of interacting with at least three or more amino acid residues selected from lysine at position 5, leucine at position 56, methionine at position 67, glutamine at position 70, tyrosine at position 71, and threonine at position 74 in the amino acid sequence set forth in SEQ ID NO: 1.

The present invention also encompasses the screening method for a Ras inhibitor candidate (a), including selecting a substance capable of interacting with the above-mentioned specified amino acid residues. In the foregoing, the interaction refers to a force between a Ras protein and a specified substance such as the Ras inhibitor candidate (a). Examples of the interaction include a hydrophilic interaction (e.g., a hydrogen bond or a salt bridge), a hydrophobic interaction (e.g., a hydrophobic bond), an electrostatic interaction, and a van der Waals interaction, preferably a hydrogen bond.

More specifically, the Ras inhibitor candidate (a) can be screened by a method including the following steps of: 1) producing a polypeptide in which at least three or more amino acid residues selected from, with reference to the amino acid sequence set forth in SEQ ID NO: 1 in the Ras partial polypeptide of the present invention, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74, preferably at least three or more amino acid residues selected from lysine at position 5, leucine at position 56, methionine at position 67, glutamine at position 70, tyrosine at position 71, and threonine at position 74 in the amino acid sequence set forth in SEQ ID NO: 1 are each labeled with an isotope measurable by NMR to obtain an NMR signal from the labeled polypeptide;

2) bringing a candidate into contact with the labeled polypeptide produced in the step 1) to obtain an NMR signal from a complex between the candidate and the labeled polypeptide; and
3) selecting a Ras inhibitor candidate (a) capable of interacting with the labeled polypeptide based on a difference between the NMR signal obtained in the step 1) and the NMR signal obtained in the step 2).

In the foregoing, the number of the amino acid residues to be each labeled with an isotope measurable by NMR is 4 or more, more preferably 6 or more.

In the foregoing, the Ras inhibitor candidate (a) is screened by selecting a substance capable of interacting with at least three or more, preferably four or more, most preferably six amino acid residues selected from, with reference to the amino acid sequence set forth in SEQ ID NO: 1, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74. The Ras inhibitor candidate (a) is preferably screened by selecting a substance capable of interacting with at least three or more, preferably four or more, most preferably six amino acid residues selected from lysine at position 5, leucine at position 56, methionine at position 67, glutamine at position 70, tyrosine at position 71, and threonine at position 74 in the amino acid sequence set forth in SEQ ID NO: 1.

This is because amino acid residues constituting the pocket present on the molecular surface of the Ras partial polypeptide are considered to include the above-mentioned amino acid residues in parts thereof. Specifically, the amino acid residues constituting the pocket present on the molecular surface of the Ras partial polypeptide are considered to include, with reference to the amino acid sequence set forth in SEQ ID NO: 1, at least lysine at position 5, leucine at position 56, methionine at position 67, glutamine at position 70, tyrosine at position 71, and threonine at position 74. The amino acid residues are considered to further include glutamic acid at position 37, aspartic acid at position 38, serine at position 39, glutamic acid at position 63, tyrosine at position 64, alanine at position 66, and arginine at position 73. The Ras inhibitor candidate (a) inhibits a Ras function through interactions with amino acid residues present at the pocket site present on the molecular surface of the Ras partial polypeptide.

In the present invention, the Ras inhibitor candidate (a) to be used in the screening method for a Ras inhibitor (A) of the present invention may be selected by a method known per se or any method to be developed in the future, not by the above-mentioned method.

Examples of the method for selection of a Ras inhibitor candidate (a) include a method involving employing X-ray crystallography of a Ras partial polypeptide. Specifically, information on a structure having a pocket of the Ras partial polypeptide is obtained by X-ray crystallography, and a Ras inhibitor candidate (a) may be designed or selected based on the structural information. In order to carry out the X-ray crystallographic analysis of the Ras partial polypeptide, as described above, it is necessary to obtain a co-crystal of the Ras partial polypeptide and GTP or a GTP analog, that is, a co-crystal of Ras-GTP. Through the use of structural information obtained from the co-crystal of Ras-GTP, a Ras inhibitor candidate (a) that binds to the pocket of the Ras partial polypeptide may be designed or selected. In order to examine the inhibitory activity of the resultant Ras inhibitor candidate (a) on Ras function, the Ras inhibitor candidate (a) may be brought into contact with Ras or the Ras partial polypeptide.

The Ras partial polypeptide for co-crystallization of Ras-GTP has only to be included in the Ras family, and may be any of three Ras isoforms, i.e., H-Ras, N-Ras, and K-Ras, and homologs such as M-Ras and Rap. A Ras protein is not particularly limited as long as it is derived from a mammal, and examples thereof include ones of human, bovine, and porcine origins. Ras is preferably, for example, H-Ras or M-Ras. As a Ras partial polypeptide most suitable for acquiring information on a structure having a pocket of Ras, there is given a Ras mutant polypeptide, that is, H-Ras T35S.

Examples of the GTP analog include guanosine 5′-(γ-thio)-triphosphate (GTPγS) and (guanosine 5′-[β,γ-imido]triphosphate (Gpp(NH)p). Of those, Gpp(NH)p is preferred. As Gpp(NH)p, for example, one purchased from CALBIOCHEM may be used.

The co-crystal of the present invention may be produced by the following method. First, a Ras-GTP solution is produced. In this case, a Ras partial polypeptide in Ras-GTP is suitably a H-Ras mutant polypeptide, that is, H-Ras T35S. Ras-GTP is allowed to exist in a solution formed of a buffer, a salt, a reducing agent, and the like. Any buffer, salt, and reducing agent may be used as long as the structure of the Ras polypeptide is not affected. Examples of the buffer include 1 to 500 mM Na-HEPES, sodium phosphate, potassium phosphate, and Tris-HCl. Examples of the salt include 1 mM to 1 M sodium chloride, lithium chloride, and magnesium chloride. Examples of the reducing agent include 0.1 to 10 mM β-mercaptoethanol and dithiothreitol (DTT). Further, the solution of the Ras partial polypeptide may contain dimethylsulfoxide (DMSO). The solution containing the Ras partial polypeptide has a pH of 4 to 11, preferably a pH of 6 to 9. Such solution of the polypeptide may be used for crystallization without any further treatment, or as necessary, a preservative, a stabilizer, a surfactant, or the like is further added to the solution, and the resultant solution may be used for crystallization.

As a crystallization method for a protein (polypeptide), a general technique for protein crystallization such as a vapor diffusion method, a batch method, or a dialysis method may be employed. Further, in the crystallization of a protein, it is important to determine physical and chemical factors such as the concentration of the protein, the concentration of a salt, a pH, the kind of a precipitant, and a temperature.

The vapor diffusion method refers to a method involving placing a droplet of a protein solution containing a precipitant in a container including a buffer (external solution) containing the precipitant at a higher concentration, sealing the container, and then leaving the resultant to stand still. The vapor diffusion method is classified into a hanging drop method and a sitting drop method depending on how to place the droplet, and any of the methods may be adopted in the present invention. The hanging drop method is a method involving placing a small droplet of a protein solution on a cover glass, inversing the cover glass in a reservoir, and sealing the reservoir. On the other hand, the sitting drop method is a method involving installing an appropriate droplet stage in a reservoir, placing a small droplet of a protein solution on the droplet stage, and sealing the reservoir with a cover glass or the like. In any of the methods, a precipitant is incorporated into the solution in the reservoir (reservoir solution). As appropriate, a small amount of the precipitant may be incorporated into a protein small droplet.

The reservoir solution to be used in the vapor diffusion method is a solution formed of a buffer, a precipitant, a salt, and the like. Any buffer, precipitant, and salt may be used as long as a crystal can be efficiently produced. Examples of the buffer include 1 to 500 mM Na-HEPES, sodium phosphate, potassium phosphate, Tris-HCl, sodium acetate, and citric acid at a pH of 4 to 9. Examples of the precipitant include 1 to 50 vol % polyethylene glycol (PEG) having a molecular weight of 400 to 10,000 or 0.2 to 3 M ammonium sulfate, 1 to 20 vol % methylpentanediol (MPD), and 5 to 10 vol % isopropanol. Examples of the salt include 0.05 to 0.3 M sodium chloride, lithium chloride, and magnesium chloride. The components for the reservoir solution are not limited to those described above.

Electron density data is obtained by a technique for crystallography with X-ray diffraction using a crystal. An electron density map may be prepared based on the electron density data. Structural information is obtained by obtaining an electron density and using a specified X-ray diffraction apparatus for a specified crystal.

The selection of a compound that may affect a pocket present on the molecular surface of the Ras partial polypeptide or a compound that has a structure capable of filling the pocket by calculation allows a Ras-specific Ras inhibitor candidate (a) to be efficiently selected from numerous candidates.

In the primary screening method, first, the structural information on the Ras partial polypeptide is used for carrying out the matching of atomic coordinates that represent a conformation having a pocket with atomic coordinates that represent a conformation of any candidate on a computer. Next, the matching state is converted into a numerical value, for example, by using an empirical scoring function as an indicator to evaluate an ability of the candidate to bind to the pocket of the Ras partial polypeptide. As atomic coordinates having the pocket of the Ras partial polypeptide, the whole atomic coordinates of the Ras partial polypeptide, derivatives thereof including the pocket, and parts thereof may be utilized. Further, atomic coordinates of a pocket part appropriately altered on a computer so as to become suitable for screening may be utilized.

A mode of a three-dimensional chemical interaction of Ras-GTP can be displayed in detail by inputting the atomic coordinates out of the structural information about the Ras partial polypeptide to a computer or a storage medium of the computer in which a computer program that displays atomic coordinates of a molecule operates. There are known a large number of commercially available computer programs that display atomic coordinates of a molecule. In general, those programs include means for inputting atomic coordinates of a molecule, means for visually displaying the coordinates on a computer screen, means for measuring a distance, a bond angle, and the like between the respective atoms in the displayed molecule, means for additionally correcting the coordinates, and the like. In addition, it is also possible to use a program including means for calculating structural energy of a molecule based on coordinates of the molecule and means for calculating free energy in consideration of a solvent molecule such as a water molecule. Computer programs InsightII and QUANTA commercially available from Accerlys are suitably used for the screening method of the present invention. However, computer programs to be used in the present invention are not limited to the above-mentioned programs.

The step of evaluating the matching state of the atomic coordinates of the candidate and the atomic coordinates having a pocket of the Ras partial polypeptide in the same coordinate system by overlapping both the coordinates can be carried out using the above-mentioned commercially available package software and a computer system capable of operating the software. The computer system appropriately includes various means necessary for operating software of interest, for example, storage means for storing a structural formula of a compound, means for converting a conformation of a compound into coordinates, storage means for storing atomic coordinates of a compound, storage means for storing atomic coordinates of Ras, storage means for storing evaluation results, means for displaying contents in each storage means, input means such as a keyboard, display means such as a display, and a central processing unit.

Any software for analysis may be used as long as the software can carry out an operation of docking a ligand to a protein on a computer, and for example, DOCK, FlexX (Tripos), LigandFit (Accerlys), Ludi (Accerlys), and the like may be used. In addition, the operation may be carried out interactively using molecular display software such as InsightII. In that case, as an indicator in evaluating the matching state using each of those programs, a free energy value calculated for the whole complex, an empirical scoring function, shape complementarity evaluation, or the like may be arbitrarily selected and used. The indicator allows whether the binding is good or bad to be objectively evaluated.

The design or selection of the Ras inhibitor candidate (a) using atomic coordinates of Ras-GTP including the Ras partial polypeptide allows quick screening on a computer.

(Ras Inhibitor Candidate (a))

The Ras inhibitor candidate (a) of the present invention is exemplified by a compound represented by the following general formula (I).

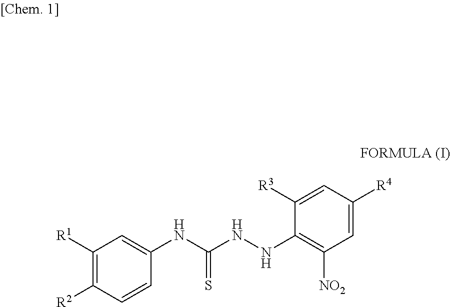

(In the formula, R¹, R², R³, and R⁴ each independently represent an atom or group selected from the group consisting of a hydrogen atom, a halogen group, a lower alkyl group (having 1 to 6 carbon atoms), a nitro group, and a trifluoromethyl group.)

Specific examples of the compound represented by the general formula (I) include compounds represented by the following formulae (II) to (IV) (Compounds (II) to (IV)).

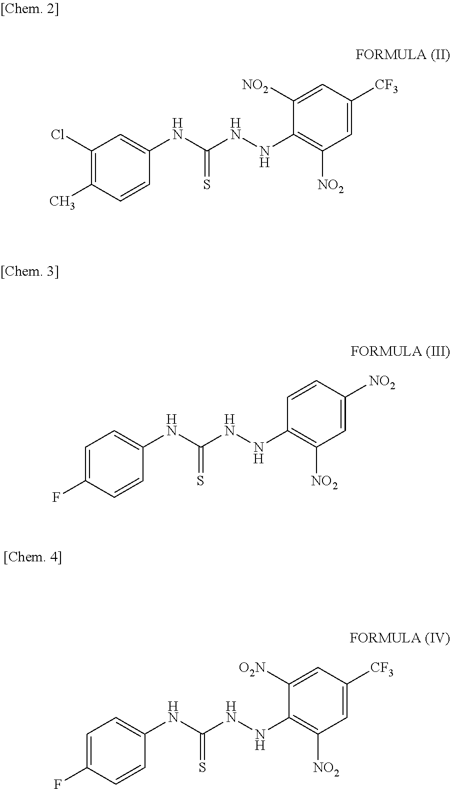

(NMR Analysis)

In carrying out the screening method for a Ras inhibitor of the present invention, a difference between an NMR signal from a Ras partial polypeptide and an NMR signal from a complex obtained by bringing a Ras inhibitor candidate (a) into contact with the Ras partial polypeptide can be analyzed as described below.

The analysis can be carried out by comparing the following NMR signals: an NMR signal (1) obtained from the Ras partial polypeptide by subjecting the Ras partial polypeptide in an aqueous solution to NMR measurement; and an NMR signal (2) obtained from the complex obtained by bringing the Ras inhibitor candidate (a) into contact with the Ras partial polypeptide by subjecting the complex to NMR measurement in the same manner as above. When the signal (2) from the complex of the Ras inhibitor candidate (a) and the Ras partial polypeptide differs from the signal (1) from the Ras partial polypeptide alone, it can be judged that the Ras inhibitor candidate (a) has some interaction with the Ras partial polypeptide. The aqueous solution may contain an organic solvent.

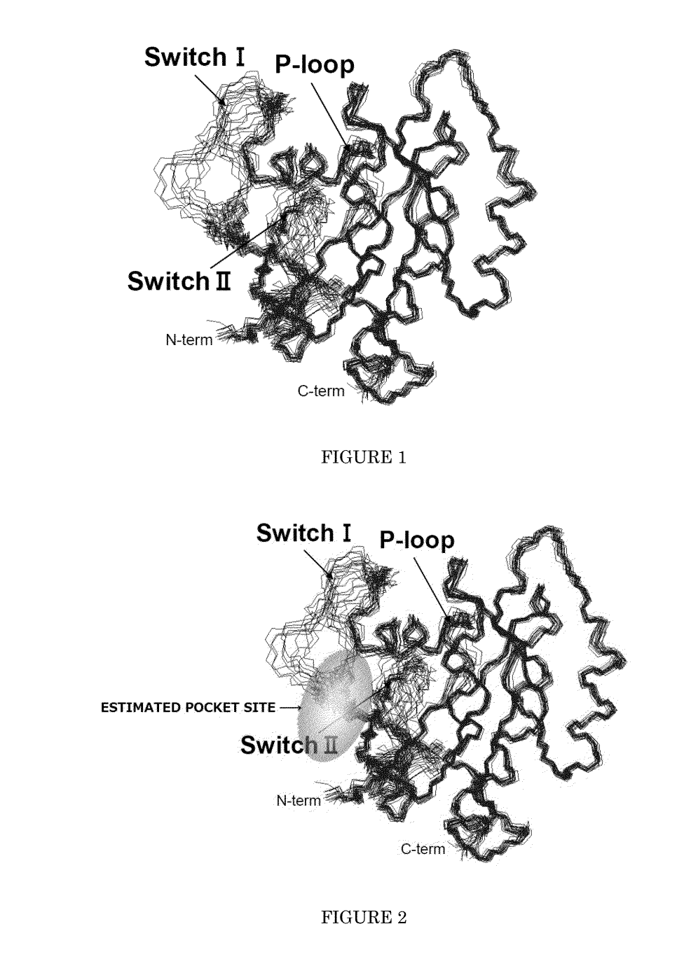

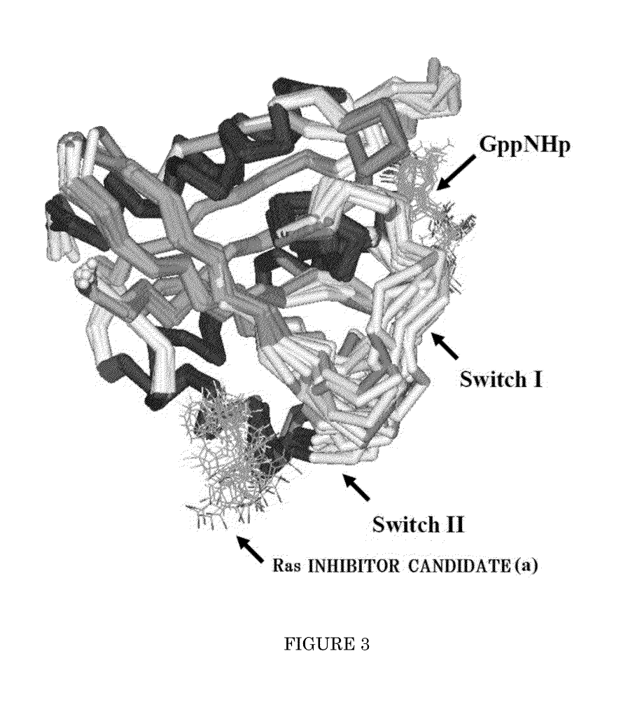

In the foregoing, the interaction refers to that a force acts between a Ras protein and a specified substance such as the Ras inhibitor candidate (a). Examples of the interaction may include a hydrophilic interaction (e.g., a hydrogen bond or a salt bridge), a hydrophobic interaction (e.g., a hydrophobic bond), an electrostatic interaction, and a van der Waals interaction. A generally employed method known per se is employed as a method of confirming a site having an interaction from an NMR signal. For example, an amino acid residue having an interaction can be identified from information based on a nuclear overhauser effect (NOE) through space, and a structural model obtained by construction based on an NMR signal through the use of, for example, a program called CONTACT in the CCP4 program suit (Collaborative Computational Project Number 4. (1994) Acta Crystallogr. D 50, 760-763), or can be judged with information obtained by visual observation. It is possible to refer to FIG. 1 for a structural model of the Ras partial polypeptide, and it is possible to refer to FIG. 2 or 3 for a structural model of the complex between the Ras inhibitor candidate (a) and the Ras partial polypeptide. It should be noted that a structural fluctuation related to a protein function is observed owing to the nature of a protein, and the structural fluctuation is also observed in each of the Ras partial polypeptide and the complex of the Ras partial polypeptide and the Ras inhibitor candidate (a). Each of FIGS. 1 to 3 shows a structural change based on a fluctuation as well.

Based on the structural information about the complex of the Ras partial polypeptide and the Ras inhibitor candidate (a), structural modification of the Ras inhibitor candidate (a) with a compound for forming a more stable complex can be carried out by visual observation on a complex model or by utilizing a of docking simulation software. In addition, a novel derivative having a higher activity and a lower toxicity can be designed and synthesized in such a form that a substituent at a high risk of expressing toxicity or a metabolically unstable chemical structure is substituted by its biological equivalent in the structure of a structure modifying compound with which the Ras inhibitor candidate (a) is modified, and the Ras inhibitor (A) that can serve as a lead compound can be derived. The Ras inhibitor (A) selected by utilizing such simulation may also be further subjected to activity and toxicity verification by an in vitro or in vivo wet assay.

In this connection, an amino acid residue of the Ras partial polypeptide with which the Ras inhibitor candidate (a) interacts can also be specified by specifying which amino acid residue a changed signal out of the above-mentioned two signals is derived from.

Specifically, in the present invention, a site having an interaction between the Ras inhibitor candidate (a) represented by the general formula (I), more specifically the Ras inhibitor candidate (a) as any one of Compounds (II) to (IV) represented by the formulae (II) to (IV) and the Ras partial polypeptide includes any one or more of lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in the amino acid sequence set forth in SEQ ID NO: 1, and for example, includes at least lysine at position 5, leucine at position 56, methionine at position 67, glutamine at position 70, tyrosine at position 71, and threonine at position 74. Further, a structural fluctuation related to a protein function is observed owing to the nature of a protein, and the structural fluctuation is also observed in the Ras partial polypeptide. Depending on fluctuations, a difference is observed in amino acid residues bound through an interaction between the Ras partial polypeptide and the Ras inhibitor candidate (a) as well. As a result of the analysis of the 15 kinds of structures obtained depending on fluctuations, any one or more of lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74, at least lysine at position 5, leucine at position 56, methionine at position 67, glutamine at position 70, tyrosine at position 71, and threonine at position 74 were found to be amino acid residues related to the interaction between the Ras partial polypeptide and the Ras inhibitor candidate (a).

Further, in the NMR measurement of the present invention, the complex between the Ras partial polypeptide and the Ras inhibitor candidate (a) can be measured in an aqueous solution containing DMSO (10 to 30%) at 3 to 10° C., preferably 5±1° C. Homonuclear multidimensional NMR measurement, heteronuclear multidimensional NMR measurement, or the like is preferably employed as a method for the NMR measurement. For example, the measurement may be carried out by an NMR measurement method called ¹H-¹⁵N HSQC. Such measurement is a technology known to a person skilled in the art. In simple terms, ¹H-¹⁵N HSQC is a correlation spectrum of a hydrogen atom and a nitrogen atom in a peptide bond in a protein, that is, a ¹H-¹⁵N correlation spectrum, and information about individual residues can be obtained from ¹H-¹⁵N signals derived from a main chain. Such NMR measurement method enables the structural analysis of a target high molecular weight substance such as a protein and enables the interaction analysis of a protein.

When the Ras partial polypeptide as a sample for the NMR measurement of the “Ras partial polypeptide” or the “complex of the Ras partial polypeptide and the Ras inhibitor candidate (a)” is produced by a technique for gene recombination, in order to facilitate the purification of the expressed polypeptide, a fused polypeptide having added thereto a tag for purification may be produced. When the Ras partial polypeptide is produced as a fused polypeptide with a protein or peptide sequence having an affinity to a specified ligand (tag for affinity purification), the fused polypeptide may be efficiently purified by affinity chromatography or the like using the ligand as a carrier. For example, as a tag for purification, there are given a 6× histidine tag, a FLAG tag, a glutathione S-transferase (GST) tag, a maltose-binding protein, and protein A. Further, the preliminary insertion of an amino acid sequence that is recognized and cleaved by a specific protease between a polypeptide of interest and a tag for purification can cleave the fused polypeptide to collect only the polypeptide of interest. For example, PreScission protease, Factor Xa, thrombin, enterokinase, collagenase, or the like may be used as the specific protease. After the cleavage with such protease, several amino acids may remain at the end of the polypeptide of interest, but the polypeptide of interest may be used as a sample for obtaining an NMR signal of the present invention as long as the amino acids do not affect an activity.

The Ras inhibitor (A) obtained by the screening method of the present invention is considered to have activities of treating and preventing various diseases that may be developed based on the aberrant Ras functions, such as an anti-cancer activity. Thus, the Ras inhibitor (A) in the present invention may be used for drugs each containing the Ras inhibitor as an active ingredient, such as therapeutic drugs for tumors including anti-cancer drugs. The anti-tumor effect of the Ras inhibitor (A) in the present invention may be evaluated by culturing cancer cells in the presence of the Ras inhibitor (A) to examine whether or not a cancer-specific phenotype of the cells is suppressed, and further using a cancer-bearing model animal.

### EXAMPLES

Hereinafter, the present invention is further specifically described by way of examples of the present invention. However, the present invention is not limited thereto, and a variety of applications thereof are possible without departing the technical idea of the present invention.

### Example 1

### With Regard to Ras Inhibitor Candidate (A)

Compounds represented by the following formulae (II) to (IV) (Compound (II), Compound (III), and Compound (IV), respectively) were each obtained as the Ras inhibitor candidate (a) of the present invention by in silico screening based on structural information in X-ray crystallography. Specifically, Compound (II) was obtained by screening with docking simulation (MMPB-SA method), and Compound (III) and Compound (IV) were each obtained by in silico screening with analogue search on Compound (II) (using the Tanimoto coefficient as an indicator). Those compounds were identified through biochemical and cytological activity verification tests shown in Experimental Examples 1 and 2 below.

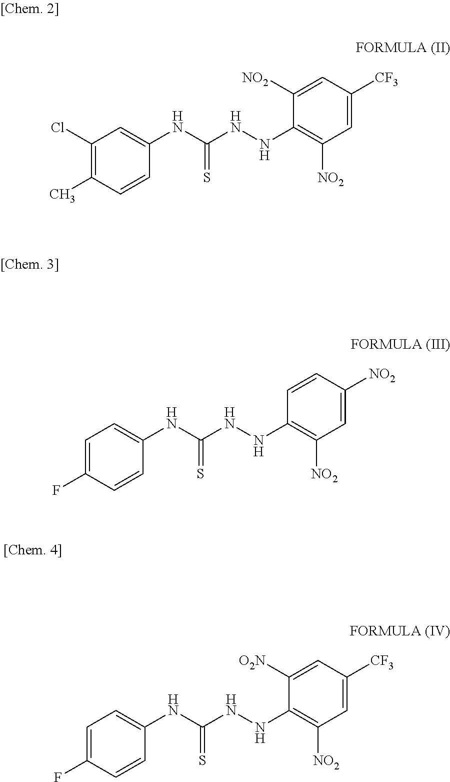 [SMILES: Cc1ccc(NC(=S)NNc2c([N+](=O)[O-])cc(C(F)(F)F)cc2[N+](=O)[O-])cc1Cl ; O=[N+]([O-])c1ccc(NNC(=S)Nc2ccc(F)cc2)c([N+](=O)[O-])c1 ; O=[N+]([O-])c1cc(C(F)(F)F)cc([N+](=O)[O-])c1NNC(=S)Nc1ccc(F)cc1]

### Experimental Example 1

### Biochemical Activity Verification Test

In this experimental example, each of Compound (II), Compound (III), and Compound (IV) obtained in Example 1 was confirmed for its Ras-Raf binding inhibition activity through a biochemical activity verification test. In this experimental example, a human H-Ras polypeptide (amino acid residues 1 to 166) was used as Ras, and a GTP analog (GTPγS (Roche Diagnostics)) was used as GTP. Further, human c-Raf-1 (amino acid residues 51 to 130) was used as Raf, a target protein of Ras.

1) GST-Raf-Fused Protein

A fused protein of Raf, a target protein of Ras, and glutathione S-transferase (GST) was expressed in Escherichia coli using a vector for GST-fused protein expression (pGEX-6P-1 vector (GE Healthcare)). The expressed GST-Raf-fused protein was immobilized onto an affinity carrier for purification (glutathione Sepharose 4B: resin (GE Healthcare)).

### 2) Radiolabeled Ras

GTPγ[³⁵S] (Perkin Elmer), one of the GTP analogs, was added to Ras from which a GST tag was cleaved with a protease for GST-fused protein cleavage (PreScission protease (GE Healthcare)) to produce radiolabeled Ras.

### 3) Measurement of Inhibitory Activities on Ras-Raf Binding

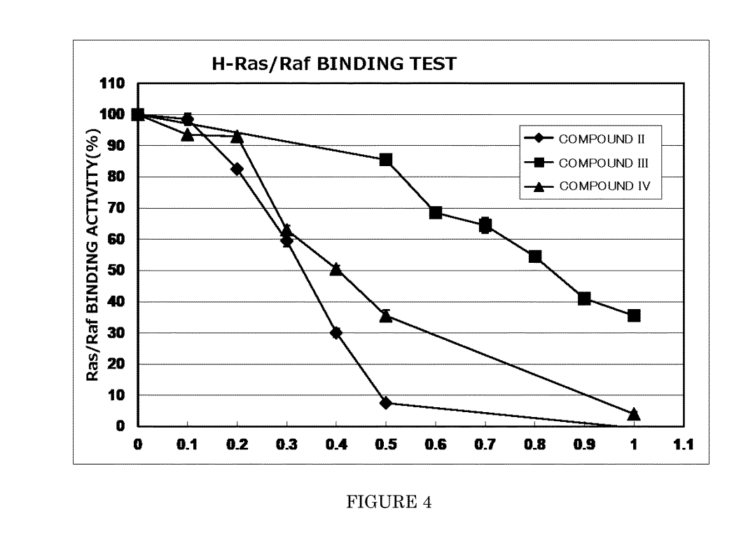

The GST-Raf-fused protein immobilized onto the resin according to the above-mentioned item 1) and the radiolabeled Ras according to the above-mentioned item 2) were incubated in the presence of the compound (Compound (II), Compound (III), or Compound (IV)) solubilized at various concentrations in 10% DMSO, to thereby promote a binding reaction of Ras to Raf. The resin in the reaction liquid was washed, and then a complex of the GST-Raf-fused protein and the radiolabeled Ras was eluted by adding glutathione. A radioactivity of the eluate was subjected to scintillation counting, to thereby measure a binding activity of Ras to Raf, and inhibitory activities on Ras-Raf binding with various compounds were evaluated. As a result, those compounds were each found to interact with Ras (FIG. 4).

### Experimental Example 2

### Cytological Activity Verification Test

In this experimental example, each of Compound (II), Compound (III), and Compound (IV) obtained in Example 1 was confirmed for its inhibitory activity on anchorage-independent cell growth through a cytological activity verification test.

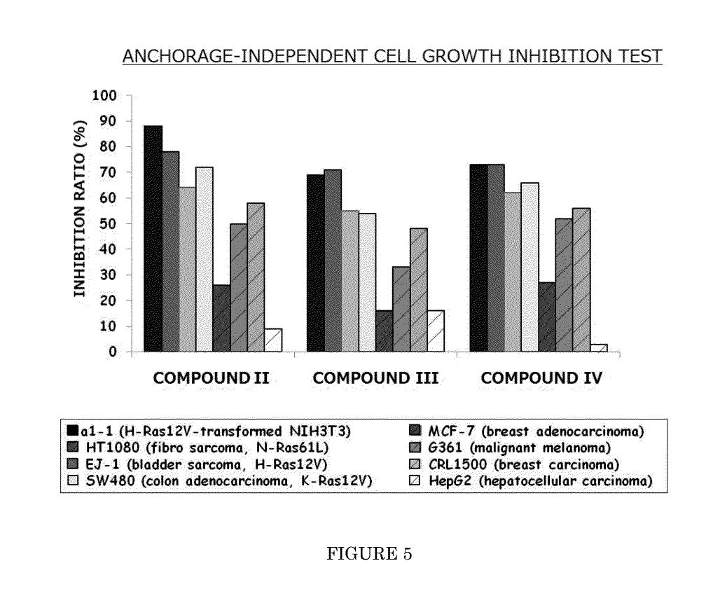

On a cell medium containing 0.6% SeaPlaque™ agarose (Lonza, Rockland, Inc., ME, USA), a medium in which each of various culture cell lines having a mutation in an active form of any one of H-Ras, N-Ras, and K-Ras was suspended, and a medium mixed with each of the compounds (Compound (II), Compound (III), or Compound (IV)) were seeded in a culture vessel, followed by culture at 37° C. for 2 weeks. The number of appeared colonies each having a size with a diameter of 50 μm or more was measured. For a medium in which each of the culture cell lines was suspended in a system free of each of the compounds, the number of colonies was similarly measured and used as a control (100%). Each of the compounds was evaluated for its inhibitory activity on colony formation (inhibitory activity on anchorage-independent cell growth) by calculating inhibition ratio for colony formation of the compound administration group relative to the control. As a result, those compounds were each found to have a colony formation inhibition effect on each cell and to interact with the three kinds of isoforms of Ras (FIG. 5).

### Example 2

### NMR Data Actually Measured for Complex of Ras and Compound (III)

In this example, NMR data actually measured for a complex of Ras and Compound (III) is shown. In this example, H-Ras T35S (amino acid residues 1 to 166), which was a H-Ras mutant, was used as Ras, and a GTP analog (Gpp(NH)p: CALBIOCHEM) was used as GTP. The same holds true for Examples shown below.

1) Production of Complex of Ras-GTP and Compound (III)

First, Ras-GTP including Ras needs to be produced in order to acquire structural information including a pocket of Ras by NMR. 3.6 mg of Ras and 1.1 mg of GTP were dissolved in 1 mL of a buffer having a neutral pH to produce Ras-GTP. Next, 3.6 mg of Ras-GTP were dissolved in 0.16 mL of a deuterated buffer solution (pH 6.8) to prepare a Ras-GTP solution. 0.3 mg of Compound (III) was dissolved in 0.04 mL of deuterated dimethyl sulfoxide (DMSO) to prepare a Compound (III) solution. The Ras-GTP solution and the Compound (III) solution were mixed with each other to produce a complex of Ras-GTP and Compound (III) under the conditions of 20% DMSO and 5° C.

2) NMR Analysis with ¹³C-Separated NOESY-HSQC Spectra

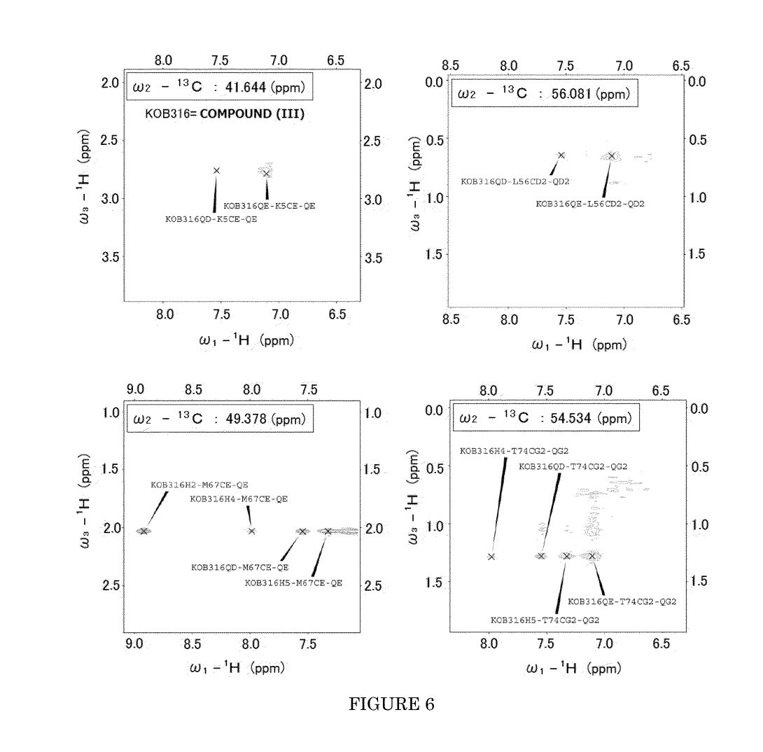

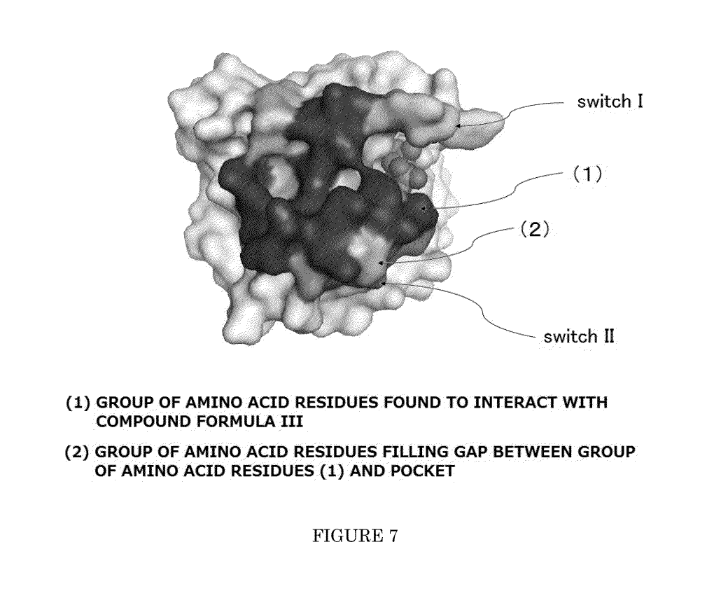

The complex of Ras-GTP and Compound (III) was subjected to NMR analysis with ¹³C-separated NOESY-HSQC spectra. In the ¹³C-separated NOESY-HSQC spectra, when specified hydrogen atoms derived from Compound (III) and Ras-GTP are spatially close to each other, a peak appears at a position where chemical shifts of both cross. The spectra are separated by chemical shifts of carbon, and hence it is possible to assign which amino acid or atomic group a hydrogen atom that provides a cross peak is derived from. Thus, according to this method, an interaction between Ras-GTP and Compound (III) can be shown directly and at an atomic level. FIG. 6 show cross peaks that appeared between hydrogen atoms derived from Compound (III) and hydrogen atoms derived from side chains of lysine at position 5, leucine at position 56, methionine at position 67, and threonine at position 74 in the polypeptide of the Ras part of Ras-GTP. It should be noted that KOB316 in FIG. 6 means Compound (III).

### Example 3

### Distance Information about Amino Acids in Proximity to Ras and Compound (III)

A complex of Ras-GTP and Compound (III) produced by the same technique as in Example 2 was confirmed for its distance information (NOE information) about amino acids in proximity to Ras and Compound (III). The complex of Ras-GTP and Compound (III) was subjected to NMR analysis with ¹³C-separated NOESY-HSQC spectra in the same manner as in Example 2. Integrated intensities of cross peaks in ¹³C-separated NOESY-HSQC depend on distances between hydrogen atoms, and hence can be converted into information about distances between Compound (III) and Ras-GTP. Table 1 shows distance information about amino acids of Ras in proximity to Compound (III) obtained based on the respective cross peaks. Specifically, the table shows information about distances between hydrogen atoms derived from side chains of amino acid residues selected from lysine at position 5, leucine at position 56, methionine at position 67, glutamine at position 70, tyrosine at position 71, and threonine at position 74 in Ras in Ras-GTP, and specified hydrogen atoms in Compound (III). The fact that a plurality of pieces of distance information is found for one kind of amino acid means that several hydrogen nuclides are present in each amino acid, and distance information corresponding to each of the nuclides is present.

[Table 1]

[symbol]

### Compound (III)

#HD, HE: hydrogen atoms derived from a benzene ring with an F atom in Compound (III)
#H2, H4, H5: hydrogen atoms derived from a benzene ring with a nitro group in Compound (III)

### Example 4

### Distance Information about Amino Acids in Proximity to Ras and Compound (III)

A complex of Ras-GTP and Compound (III) (kobe2601) produced by the same technique as in Example 2 was subjected to calculation with a program called CONTACT in the CCP4 program suit (Collaborative Computational Project Number 4. (1994) Acta Crystallogr. D 50, 760-763) based on 15 kinds of NMR structures of the complex. As a result, a group of residues (1), which were able to be found to interact with Compound (III), and a group of residues (2), which were selected so as to fill a gap in a pocket, are shown below. With reference to the amino acid sequence set forth in SEQ ID NO: 1, the group (1) consists of lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74, and the group (2) consists of leucine (L) at position 6, valine (V) at position 7, isoleucine (I) at position 36, arginine (R) at position 41, aspartic acid (D) at position 54, isoleucine (I) at position 55, aspartic acid (D) at position 57, threonine (T) at position 58, alanine (A) at position 59, serine (S) at position 65, and glycine (G) at position 75. Hereinafter, Tables 2 to Tables 16 show CONTACT information calculated for the 15 kinds of complex models, respectively.

TABLE 2
model01

| kobe2601 | H-RasT35S-GppNHp | | | | |
|---|---|---|---|---|---|
| atom | residue | number | atom | distance | angle |
| C13 | Tyr | 71 | HE2 | 2.41 | |
| | Tyr | 71 | CE2 | 3.48 | |
| C12 | Tyr | 71 | HE2 | 2.46 | |
| | Glu | 37 | HG1 | 3.42 | |
| | Tyr | 71 | CE2 | 3.38 | |
| C11 | Leu | 56 | HD12 | 3.48 | |
| | Lys | 5 | HE1 | 3.27 | |
| | Tyr | 71 | HE2 | 3.25 | |
| | Leu | 56 | HD13 | 3.53 | |
| F11 | Leu | 56 | HD12 | 2.75 | |
| | Lys | 5 | CE | 3.58 | |
| | Lys | 5 | HE1 | 2.56 | |
| | Leu | 56 | CD1 | 3.07 | |
| | Leu | 56 | HD11 | 3.47 | |
| | Leu | 56 | HD13 | 2.58 | |
| C10 | Lys | 5 | HE1 | 3.19 | |
| | Lys | 5 | HZ3 | 3.37 | |
| CB | Tyr | 71 | HE2 | 3.17 | |
| N8 | Gln | 70 | HG2 | 3.49 | |
| | Gln | 70 | HE21 | 3.50 | |
| C7 | Gln | 70 | HG2 | 3.44 | |
| | Gln | 70 | NE2 | 3.41 | |
| | Gln | 70 | HE21 | 2.58 | |
| S7 | Gln | 70 | NE2 | 3.33 | |
| | Gln | 70 | HE21 | 2.40 | |
| N7 | Gln | 70 | HE22 | 3.53 | |
| | Gln | 70 | NE2 | 3.30 | *** |
| | Gln | 70 | HE21 | 2.75 | |
| HAC | Gln | 70 | HE22 | 3.00 | |
| | Gln | 70 | NE2 | 3.03 | |
| | Gln | 70 | HE21 | 2.64 | |
| C5 | Gln | 70 | HE22 | 3.30 | |
| | Gln | 70 | CG | 3.36 | |
| | Gln | 70 | HG2 | 2.78 | |
| | Gln | 70 | CD | 3.04 | |
| | Gln | 70 | NE2 | 2.94 | |
| | Gln | 70 | HE21 | 3.01 | |
| C4 | Gln | 70 | OE1 | 3.45 | |
| | Gln | 70 | HB2 | 3.12 | |
| | Gln | 70 | CG | 3.53 | |
| | Gln | 70 | HG2 | 3.17 | |
| | Gln | 70 | CD | 3.28 | |
| | Met | 67 | HE3 | 3.14 | |
| C3 | Met | 67 | CE | 2.97 | |
| | Met | 67 | HE1 | 2.88 | |
| | Met | 67 | HE2 | 3.07 | |
| | Met | 67 | HE3 | 2.48 | |
| N3 | Ala | 66 | O | 3.35 | * |
| | Ala | 66 | HB1 | 3.02 | |
| | Met | 67 | CE | 2.88 | |
| | Met | 67 | HE1 | 2.81 | |
| | Met | 67 | HE2 | 2.55 | |
| | Met | 67 | HE3 | 2.81 | |
| O3B | Ala | 66 | CB | 3.38 | |
| | Ala | 66 | HB1 | 2.74 | |
| | Ala | 66 | HB2 | 3.07 | |
| | Met | 67 | CE | 2.76 | |
| | Met | 67 | HE1 | 2.31 | |
| | Met | 67 | HE2 | 2.43 | |
| | Met | 67 | HE3 | 3.16 | |
| O3A | Ala | 66 | C | 3.02 | |
| | Ala | 66 | O | 2.33 | *** |
| | Gln | 70 | HB2 | 3.43 | |
| | Ala | 66 | CB | 3.36 | |
| | Ala | 66 | HB1 | 2.50 | |
| | Met | 67 | HE2 | 3.14 | |
| C2 | Met | 67 | CE | 2.95 | |
| | Met | 67 | HE1 | 2.50 | |
| | Met | 67 | HE2 | 3.46 | |
| | Met | 67 | HE3 | 2.54 | |
| C1 | Met | 67 | HE1 | 3.53 | |
| | Met | 67 | HE3 | 3.23 | |
| H2 | Met | 67 | CE | 2.25 | |
| | Met | 67 | HE1 | 2.04 | |
| | Met | 67 | HE2 | 3.38 | |
| | Met | 67 | HE3 | 2.82 | |
| H4 | Gln | 70 | OE1 | 2.66 | |
| | Ala | 66 | O | 3.57 | |
| | Gln | 70 | CB | 2.92 | |
| | Gln | 70 | HB1 | 3.23 | |
| | Gln | 70 | HB2 | 2.17 | |
| | Gln | 70 | CG | 3.02 | |
| | Gln | 70 | HG2 | 2.98 | |
| | Gln | 70 | CD | 2.75 | |
| | Gln | 70 | NE2 | 3.49 | |
| H5 | Gln | 70 | OE1 | 3.10 | |
| | Gln | 70 | HE22 | 2.59 | |
| | Gln | 70 | CB | 3.57 | |
| | Gln | 70 | HB2 | 3.52 | |
| | Gln | 70 | CG | 2.55 | |
| | Gln | 70 | HG1 | 3.35 | |
| | Gln | 70 | HG2 | 2.01 | |
| | Gln | 70 | CD | 2.24 | |
| | Gln | 70 | NE2 | 2.02 | |
| | Gln | 70 | HE21 | 1.98 | |
| HD1 | Gln | 70 | HE21 | 3.24 | |
| | Thr | 74 | HG21 | 3.18 | |
| | Thr | 74 | HG23 | 3.56 | |
| HE1 | Thr | 74 | HG21 | 3.54 | |
| | Lys | 5 | CE | 3.09 | |
| | Lys | 5 | HE1 | 2.45 | |
| | Lys | 5 | HE2 | 3.25 | |
| | Lys | 5 | NZ | 3.16 | |
| | Lys | 5 | HZ3 | 2.45 | |
| | Thr | 74 | HB | 3.29 | |
| HE2 | Tyr | 71 | HE2 | 2.46 | |
| | Glu | 37 | CG | 3.31 | |
| | Glu | 37 | HG1 | 2.56 | |
| | Glu | 37 | CD | 3.15 | |
| | Glu | 37 | OE2 | 3.29 | |
| | Leu | 56 | CD1 | 3.42 | |
| | Leu | 56 | HD11 | 2.97 | |
| | Leu | 56 | HD13 | 3.13 | |
| | Tyr | 71 | CE2 | 3.18 | |
| HD2 | Tyr | 71 | HE2 | 2.41 | |
| | Tyr | 71 | OH | 3.54 | |
| | Glu | 37 | HG1 | 3.57 | |
| | Tyr | 71 | CE2 | 3.41 | |

TABLE 3
model02

| kobe2601 | H-RasT35S-GppNHp | | | | |
|---|---|---|---|---|---|
| atom | residue | number | atom | distance | angle |
| C13 | Tyr | 71 | CE1 | 3.53 | |
| | Tyr | 71 | HE1 | 2.72 | |
| C12 | Tyr | 71 | HE1 | 2.94 | |
| C11 | Leu | 56 | CD1 | 3.26 | |
| | Leu | 56 | HD11 | 3.42 | |
| | Leu | 56 | HD12 | 2.97 | |
| | Leu | 56 | HD13 | 2.86 | |
| | Tyr | 71 | HE1 | 2.94 | |
| | Lys | 5 | HE1 | 2.73 | |
| | Lys | 5 | HZ3 | 3.24 | |
| F11 | Lys | 5 | CE | 3.16 | |
| | Lys | 5 | HD1 | 3.56 | |
| | Leu | 56 | CD1 | 3.35 | |
| | Leu | 56 | HD12 | 3.30 | |
| | Lou | 56 | HD13 | 2.58 | |
| | Lys | 5 | HE1 | 2.54 | |
| | Lys | 5 | NZ | 2.93 | |
| | Lys | 5 | HZ2 | 2.65 | |
| | Lys | 5 | HZ3 | 2.64 | |
| C10 | Thr | 74 | OG1 | 3.15 | |
| | Thr | 74 | HG1 | 3.04 | |
| | Lys | 5 | CE | 3.49 | |
| | Thr | 74 | HB | 3.50 | |
| | Leu | 56 | CD1 | 3.19 | |
| | Leu | 56 | HD11 | 3.48 | |
| | Leu | 56 | HD12 | 2.46 | |
| | Lou | 56 | HD13 | 3.17 | |
| | Tyr | 71 | CD1 | 3.32 | |
| | Tyr | 71 | HD1 | 2.63 | |
| | Tyr | 71 | CE1 | 3.36 | |
| | Tyr | 71 | HE1 | 2.72 | |
| | Lys | 5 | HE1 | 2.42 | |
| C9 | Gln | 70 | HG1 | 3.13 | |
| | Tyr | 71 | HA | 3.24 | |
| | Thr | 74 | OG1 | 2.92 | |
| | Thr | 74 | HG1 | 3.20 | |
| | Thr | 74 | HG21 | 2.93 | |
| | Leu | 56 | HD12 | 3.12 | |
| | Tyr | 71 | CD1 | 2.81 | |
| | Tyr | 71 | HD1 | 2.42 | |
| | Tyr | 71 | CE1 | 2.84 | |
| | Tyr | 71 | HE1 | 2.47 | |
| | Lys | 5 | HE1 | 3.55 | |
| C8 | Gln | 70 | HG1 | 3.33 | |
| | Thr | 74 | HG21 | 3.41 | |
| | Gln | 70 | HG2 | 3.20 | |
| | Tyr | 71 | CD1 | 3.43 | |
| | Tyr | 71 | HD1 | 3.43 | |
| | Tyr | 71 | CE1 | 2.94 | |
| | Tyr | 71 | HE1 | 2.46 | |
| N8 | Gln | 70 | CG | 2.88 | |
| | Gln | 70 | HG1 | 2.78 | |
| | Thr | 74 | HG21 | 3.50 | |
| | Gln | 70 | HB1 | 3.34 | |
| | Gln | 70 | HG2 | 2.13 | |
| | Gln | 70 | HE21 | 3.49 | |
| | Tyr | 71 | CE1 | 3.31 | |
| | Tyr | 71 | HE1 | 3.20 | |
| HAD | Gln | 70 | CB | 2.81 | |
| | Gln | 70 | HB2 | 3.58 | |
| | Gln | 70 | CG | 2.14 | |
| | Gln | 70 | HG1 | 1.99 | |
| | Gln | 70 | CD | 3.51 | |
| | Thr | 74 | HG21 | 3.19 | |
| | Gln | 70 | HB1 | 2.45 | |
| | Gln | 70 | HG2 | 1.65 | |
| | Gln | 70 | HE21 | 3.46 | |
| | Tyr | 71 | CD1 | 3.53 | |
| | Tyr | 71 | CE1 | 3.42 | |
| | Tyr | 71 | HE1 | 3.59 | |
| C7 | Gln | 70 | CG | 3.47 | |
| | Gln | 70 | HG2 | 2.46 | |
| N7 | Gln | 70 | CG | 3.32 | |
| | Met | 67 | HA | 3.31 | |
| | Met | 67 | CE | 3.40 | |
| | Met | 67 | HE3 | 2.50 | |
| | Gln | 70 | HB1 | 3.18 | |
| | Gln | 70 | HG2 | 2.34 | |
| HAC | Gln | 70 | CB | 2.71 | |
| | Gln | 70 | HB2 | 2.81 | |
| | Gln | 70 | CG | 2.70 | |
| | Gln | 70 | HG1 | 3.37 | |
| | Met | 67 | HA | 2.73 | |
| | Met | 67 | CE | 3.45 | |
| | Met | 67 | HE3 | 2.40 | |
| | Gln | 70 | HB1 | 2.23 | |
| | Gln | 70 | HG2 | 1.92 | |
| N6 | Met | 67 | SD | 3.43 | |
| | Met | 67 | CE | 2.75 | |
| | Met | 67 | HE1 | 2.69 | |
| | Met | 67 | HE3 | 2.12 | |
| | Gln | 70 | HG2 | 3.38 | |
| HAB | Met | 67 | SD | 2.90 | |
| | Met | 67 | CE | 2.67 | |
| | Met | 67 | HE1 | 2.53 | |
| | Met | 67 | HE3 | 2.38 | |
| C6 | Met | 67 | CE | 2.93 | |
| | Met | 67 | HE1 | 2.56 | |
| | Met | 67 | HE3 | 2.39 | |
| C5 | Gln | 70 | HB2 | 3.12 | |
| | Met | 67 | HE1 | 3.59 | |
| | Met | 67 | HE3 | 3.01 | |
| | Gln | 70 | HG2 | 3.10 | |
| C2 | Met | 67 | HE1 | 3.19 | |
| C1 | Met | 67 | CE | 3.11 | |
| | Met | 67 | HE1 | 2.28 | |
| | Met | 67 | HE3 | 3.04 | |
| N1 | Met | 67 | CE | 3.12 | |
| | Met | 67 | HE1 | 2.15 | |
| | Met | 67 | HE3 | 3.39 | |
| O1B | Met | 67 | SD | 3.07 | |
| | Met | 67 | CE | 2.96 | |
| | Met | 67 | HE1 | 2.33 | |
| | Met | 67 | HE3 | 3.22 | |
| O1A | Met | 67 | HE1 | 3.03 | |
| H2 | Met | 67 | HE1 | 3.48 | |
| F14 | Gln | 70 | HB2 | 3.42 | |
| | Arg | 73 | HH21 | 3.48 | |
| | Arg | 73 | HH22 | 3.58 | |
| H5 | Gln | 70 | CB | 2.67 | |
| | Gln | 70 | HB2 | 2.15 | |
| | Gln | 70 | CG | 2.70 | |
| | Gln | 70 | CD | 3.11 | |
| | Gln | 70 | NE2 | 3.39 | |
| | Met | 67 | HE3 | 3.08 | |
| | Gln | 70 | HB1 | 2.80 | |
| | Gln | 70 | HG2 | 2.19 | |
| | Gln | 70 | HE21 | 3.27 | |
| HD1 | Gln | 70 | CG | 3.43 | |
| | Gln | 70 | HG1 | 2.51 | |
| | Tyr | 71 | N | 3.32 | |
| | Tyr | 71 | CA | 3.13 | |
| | Tyr | 71 | HA | 2.24 | |
| | Thr | 74 | OG1 | 2.28 | |
| | Thr | 74 | HG1 | 2.69 | |
| | Thr | 74 | CB | 3.29 | |
| | Thr | 74 | HB | 3.58 | |
| | Thr | 74 | CG2 | 3.38 | |
| | Thr | 74 | HG21 | 2.70 | |
| | Gln | 70 | HG2 | 3.49 | |
| | Leu | 56 | HD12 | 3.33 | |
| | Tyr | 71 | CG | 3.30 | |
| | Tyr | 71 | CD1 | 2.42 | |
| | Tyr | 71 | HD1 | 2.01 | |
| | Tyr | 71 | CE1 | 2.91 | |
| | Tyr | 71 | HE1 | 2.93 | |
| HE1 | Tyr | 71 | HA | 3.43 | |
| | Thr | 74 | OG1 | 2.82 | |
| | Thr | 74 | HG1 | 2.41 | |
| | Lys | 5 | HD2 | 3.19 | |
| | Lys | 5 | CE | 3.13 | |
| | Thr | 74 | HB | 3.34 | |
| | Lys | 5 | CD | 3.56 | |
| | Leu | 56 | CD1 | 2.92 | |
| | Leu | 56 | HD11 | 3.51 | |
| | Leu | 56 | HD12 | 2.05 | |
| | Leu | 56 | HD13 | 2.96 | |
| | Tyr | 71 | CD1 | 3.29 | |
| | Tyr | 71 | HD1 | 2.36 | |
| | Tyr | 71 | HE1 | 3.21 | |
| | Lys | 5 | HE1 | 2.11 | |
| HE2 | Tyr | 71 | HE1 | 3.55 | |
| HD2 | Tyr | 71 | HE1 | 3.24 | |

TABLE 4
model03

| kobe2601 | H-RasT35S-GppNHp | | | | |
|---|---|---|---|---|---|
| atom | residue | number | atom | distance | angle |
| C13 | Gln | 70 | HE21 | 2.95 | |
| C12 | Gln | 70 | HE21 | 3.29 | |
| C11 | Thr | 74 | CG2 | 3.58 | |
| | Thr | 74 | HG22 | 3.11 | |
| | Thr | 74 | HG23 | 3.49 | |
| | Thr | 74 | HB | 2.89 | |
| | Lys | 5 | CE | 3.57 | |
| | Lys | 5 | HE1 | 2.83 | |
| | Lys | 5 | NZ | 3.52 | |
| | Lys | 5 | HZ3 | 2.91 | |
| F11 | Thr | 74 | CB | 3.55 | |
| | Thr | 74 | HG22 | 3.18 | |
| | Thr | 74 | HB | 2.59 | |
| | Lys | 5 | CE | 3.21 | |
| | Lys | 5 | HE1 | 2.52 | |
| | Lys | 5 | HE2 | 3.21 | |
| | Lys | 5 | NZ | 3.57 | |
| | Lys | 5 | HZ3 | 3.01 | |
| C10 | Thr | 74 | CB | 3.22 | |
| | Thr | 74 | CG2 | 3.45 | |
| | Thr | 74 | HG22 | 3.42 | |
| | Thr | 74 | HG23 | 3.14 | |
| | Thr | 74 | HB | 2.74 | |
| | Tyr | 71 | HD1 | 3.44 | |
| | Thr | 74 | OG1 | 3.02 | |
| | Thr | 74 | HG1 | 2.67 | |
| | Lys | 5 | CE | 3.24 | |
| | Lys | 5 | HE1 | 2.45 | |
| | Lys | 5 | NZ | 3.16 | |
| | Lys | 5 | HZ2 | 2.91 | |
| | Lys | 5 | HZ3 | 2.89 | |
| C9 | Thr | 74 | HG23 | 3.32 | |
| | Tyr | 71 | CD1 | 3.10 | |
| | Tyr | 71 | HD1 | 2.84 | |
| | Tyr | 71 | CE1 | 2.94 | |
| | Tyr | 71 | HE1 | 2.53 | |
| | Thr | 74 | OG1 | 3.53 | |
| | Thr | 74 | HG1 | 3.31 | |
| | Lys | 5 | HZ2 | 3.37 | |
| C8 | Gln | 70 | HE21 | 3.44 | |
| | Tyr | 71 | CE1 | 3.37 | |
| | Tyr | 71 | HE1 | 2.97 | |
| N8 | Tyr | 71 | CE1 | 2.93 | |
| | Tyr | 71 | HE1 | 2.76 | |
| | Tyr | 71 | CZ | 3.11 | |
| | Tyr | 71 | OH | 3.15 | *** |
| HAD | Tyr | 71 | CD1 | 2.96 | |
| | Tyr | 71 | HD1 | 3.56 | |
| | Tyr | 71 | CE1 | 2.05 | |
| | Tyr | 71 | HE1 | 2.13 | |
| | Tyr | 71 | CE2 | 3.06 | |
| | Glu | 37 | OE2 | 3.50 | |
| | Tyr | 71 | CZ | 2.13 | |
| | Tyr | 71 | OH | 2.37 | |
| | Tyr | 71 | HH | 3.30 | |
| C7 | Tyr | 71 | OH | 3.51 | |
| S7 | Gln | 70 | HE21 | 3.53 | |
| N7 | Met | 67 | HB2 | 3.16 | |
| | Met | 67 | CE | 3.21 | |
| | Met | 67 | HE1 | 2.40 | |
| | Met | 67 | HE2 | 3.16 | |
| | Tyr | 71 | OH | 3.08 | *** |
| HAC | Tyr | 71 | CE2 | 3.37 | |
| | Met | 67 | CB | 3.50 | |
| | Met | 67 | HB1 | 3.59 | |
| | Met | 67 | HB2 | 2.62 | |
| | Met | 67 | SD | 3.38 | |
| | Met | 67 | CE | 2.82 | |
| | Met | 67 | HE1 | 2.14 | |
| | Met | 67 | HE2 | 2.85 | |
| | Tyr | 71 | HE2 | 3.33 | |
| | Tyr | 71 | CZ | 2.88 | |
| | Tyr | 71 | OH | 2.29 | |
| | Tyr | 71 | HH | 2.79 | |
| N6 | Met | 67 | CE | 2.86 | |
| | Met | 67 | HE3 | 3.47 | |
| | Met | 67 | HE1 | 2.15 | |
| | Met | 67 | HE2 | 2.71 | |
| HAB | Met | 67 | CE | 2.89 | |
| | Met | 67 | HE3 | 3.46 | |
| | Met | 67 | HE1 | 2.52 | |
| | Met | 67 | HE2 | 2.38 | |
| C6 | Met | 67 | HB1 | 3.37 | |
| | Met | 67 | CE | 3.23 | |
| | Met | 67 | HE3 | 3.50 | |
| | Met | 67 | HE1 | 2.39 | |
| | Met | 67 | HE2 | 3.39 | |
| C5 | Met | 67 | HB1 | 3.09 | |
| | Met | 67 | HB2 | 3.32 | |
| | Met | 67 | HE1 | 2.91 | |
| C4 | Met | 67 | HB1 | 3.52 | |
| C1 | Met | 67 | HE3 | 3.58 | |
| | Met | 67 | HE1 | 3.14 | |
| N1 | Met | 67 | HE1 | 3.57 | |
| | Met | 67 | HE2 | 3.51 | |
| O1B | Met | 67 | CE | 3.46 | |
| | Met | 67 | HE1 | 3.41 | |
| | Met | 67 | HE2 | 2.82 | |
| H4 | Gln | 70 | OE1 | 3.01 | |
| | Met | 67 | HA | 3.39 | |
| | Gln | 70 | CD | 3.22 | |
| | Gln | 70 | NE2 | 3.38 | |
| | Gln | 70 | HE22 | 3.27 | |
| H5 | Met | 67 | HA | 3.58 | |
| | Gln | 70 | NE2 | 3.37 | |
| | Gln | 70 | HE21 | 3.35 | |
| | Met | 67 | HB1 | 3.35 | |
| | Met | 67 | HB2 | 3.00 | |
| | Met | 67 | HE1 | 3.19 | |
| HD1 | Thr | 74 | HG23 | 3.55 | |
| | Leu | 56 | HG | 3.32 | |
| | Leu | 56 | HD23 | 3.07 | |
| | Tyr | 71 | HA | 3.05 | |
| | Tyr | 71 | CG | 3.19 | |
| | Tyr | 71 | CD1 | 2.02 | |
| | Tyr | 71 | HD1 | 1.83 | |
| | Tyr | 71 | CE1 | 2.06 | |
| | Tyr | 71 | HE1 | 1.90 | |
| | Thr | 74 | OG1 | 3.37 | |
| | Thr | 74 | HG1 | 3.13 | |
| | Lys | 5 | HZ2 | 3.52 | |
| | Tyr | 71 | CZ | 3.23 | |
| HE1 | Thr | 74 | CB | 2.93 | |
| | Thr | 74 | HG23 | 3.48 | |
| | Thr | 74 | HB | 2.39 | |
| | Leu | 56 | HG | 3.42 | |
| | Tyr | 71 | HD1 | 3.19 | |
| | Thr | 74 | OG1 | 2.56 | |
| | Thr | 74 | HG1 | 1.93 | |
| | Lys | 5 | CD | 3.28 | |
| | Lys | 5 | HD2 | 2.99 | |
| | Lys | 5 | CE | 2.47 | |
| | Lys | 5 | HE1 | 1.62 | |
| | Lys | 5 | HE2 | 3.30 | |
| | Lys | 5 | NZ | 2.71 | |
| | Lys | 5 | HZ2 | 2.45 | |
| | Lys | 5 | HZ3 | 2.79 | |
| HD2 | Gln | 70 | HE21 | 3.18 | |

TABLE 5
model04

| kobe2601 | H-RasT35S-GppNHp | | | | |
|---|---|---|---|---|---|
| atom | residue | number | atom | distance | angle |
| C12 | Lys | 5 | HZ3 | 3.35 | |
| | Lys | 5 | HZ1 | 3.58 | |
| C11 | Thr | 74 | HB | 3.13 | |
| | Lys | 5 | HE1 | 3.17 | |
| | Lys | 5 | CE | 3.41 | |
| | Lys | 5 | HE2 | 3.34 | |
| | Lys | 5 | HZ3 | 2.64 | |
| | Lys | 5 | NZ | 3.10 | |
| | Lys | 5 | HZ1 | 2.91 | |
| F11 | Thr | 74 | HB | 3.08 | |
| | Lys | 5 | HE1 | 3.12 | |
| | Lys | 5 | CE | 3.06 | |
| | Lys | 5 | HE2 | 2.59 | |
| | Lys | 5 | HZ3 | 3.04 | |
| | Lys | 5 | NZ | 3.04 | |
| | Lys | 5 | HZ1 | 2.60 | |
| C10 | Thr | 74 | CB | 3.03 | |
| | Thr | 74 | HB | 2.45 | |
| | Thr | 74 | OG1 | 2.94 | |
| | Thr | 74 | CG2 | 3.37 | |
| | Thr | 74 | HG21 | 2.95 | |
| | Thr | 74 | HG23 | 3.58 | |
| | Lys | 5 | HE1 | 2.56 | |
| | Thr | 74 | HG1 | 2.61 | |
| | Lys | 5 | CE | 3.20 | |
| | Lys | 5 | HE2 | 3.45 | |
| | Lys | 5 | HZ3 | 2.49 | |
| | Lys | 5 | NZ | 3.16 | |
| | Lys | 5 | HZ1 | 3.42 | |
| C9 | Gln | 70 | HG1 | 3.24 | |
| | Thr | 74 | HB | 3.47 | |
| | Thr | 74 | OG1 | 3.24 | |
| | Thr | 74 | HG21 | 2.93 | |
| | Lys | 5 | HE1 | 3.56 | |
| | Thr | 74 | HG1 | 3.07 | |
| | Lys | 5 | HZ3 | 3.11 | |
| C7 | Gln | 70 | HG1 | 3.34 | |
| | Met | 67 | HE1 | 3.46 | |
| | Gln | 70 | HG2 | 3.06 | |
| | Tyr | 71 | CZ | 3.44 | |
| | Tyr | 71 | CE1 | 3.23 | |
| | Tyr | 71 | HE1 | 3.31 | |
| S7 | Tyr | 71 | HN | 3.29 | |
| | Gln | 70 | CB | 3.58 | |
| | Gln | 70 | CG | 3.07 | |
| | Gln | 70 | HG1 | 2.38 | |
| | Gln | 70 | C | 3.55 | |
| | Tyr | 71 | N | 3.15 | |
| | Thr | 74 | HG21 | 3.57 | |
| | Tyr | 71 | CA | 3.34 | |
| | Tyr | 71 | HA | 2.76 | |
| | Gln | 70 | HB1 | 3.14 | |
| | Gln | 70 | HG2 | 3.00 | |
| | Tyr | 71 | CG | 3.07 | |
| | Tyr | 71 | CD2 | 3.55 | |
| | Tyr | 71 | CZ | 3.49 | |
| | Tyr | 71 | CD1 | 2.74 | |
| | Tyr | 71 | HD1 | 3.01 | |
| | Tyr | 71 | CE1 | 2.98 | |
| | Tyr | 71 | HE1 | 3.40 | |
| N7 | Met | 67 | HB1 | 3.12 | |
| | Met | 67 | CE | 3.11 | |
| | Met | 67 | HE1 | 2.14 | |
| | Met | 67 | HE2 | 3.56 | |
| | Gln | 70 | HG2 | 3.40 | |
| | Tyr | 71 | CZ | 3.24 | |
| | Tyr | 71 | OH | 3.28 | *** |
| | Tyr | 71 | CE1 | 3.57 | |
| | Met | 67 | HE3 | 3.32 | |
| HAC | Met | 67 | CB | 3.22 | |
| | Met | 67 | HB1 | 2.16 | |
| | Met | 67 | CE | 2.76 | |
| | Met | 67 | HE1 | 1.70 | |
| | Met | 67 | HE2 | 3.19 | |
| | Gln | 70 | HB1 | 3.42 | |
| | Gln | 70 | HG2 | 3.52 | |
| | Tyr | 71 | CE2 | 3.07 | |
| | Tyr | 71 | HE2 | 324 | |
| | Tyr | 71 | CZ | 2.98 | |
| | Tyr | 71 | OH | 3.18 | |
| | Tyr | 71 | CE1 | 3.57 | |
| | Met | 67 | HE3 | 3.29 | |
| N6 | Met | 67 | CE | 2.69 | |
| | Met | 67 | HE1 | 2.05 | |
| | Met | 67 | HE2 | 3.19 | |
| | Tyr | 71 | OH | 3.58 | * |
| | Met | 67 | HE3 | 2.52 | |
| HAB | Met | 67 | CE | 2.77 | |
| | Met | 67 | HE1 | 2.40 | |
| | Met | 67 | HE2 | 2.97 | |
| | Tyr | 71 | OH | 3.13 | |
| | Met | 67 | HE3 | 2.51 | |
| | Glu | 37 | HG1 | 3.05 | |
| | Glu | 37 | HG2 | 3.20 | |
| C6 | Met | 67 | CE | 2.96 | |
| | Met | 67 | HE1 | 2.53 | |
| | Met | 67 | HE3 | 2.46 | |
| C5 | Met | 67 | HE1 | 3.06 | |
| | Gln | 70 | HG2 | 2.88 | |
| | Met | 67 | HE3 | 3.36 | |
| C4 | Gln | 70 | HG2 | 3.53 | |
| C2 | Met | 67 | HE3 | 3.42 | |
| C1 | Met | 67 | CE | 3.40 | |
| | Met | 67 | HE1 | 3.39 | |
| | Met | 67 | HE3 | 2.51 | |
| N1 | Met | 67 | CE | 3.57 | |
| | Met | 67 | HE3 | 2.61 | |
| O1B | Met | 67 | CE | 3.36 | |
| | Met | 67 | HE1 | 3.47 | |
| | Met | 67 | HE2 | 3.42 | |
| | Met | 67 | HE3 | 2.68 | |
| | Glu | 37 | CG | 3.52 | |
| | Glu | 37 | HG1 | 3.38 | |
| | Glu | 37 | HG2 | 2.75 | |
| O1A | Met | 67 | HE3 | 3.59 | |
| H4 | Gln | 70 | HG2 | 3.29 | |
| H5 | Gln | 70 | CB | 3.50 | |
| | Gln | 70 | CG | 2.97 | |
| | Gln | 70 | HG1 | 3.49 | |
| | Met | 67 | HE1 | 3.06 | |
| | Gln | 70 | HB1 | 3.21 | |
| | Gln | 70 | HG2 | 1.92 | |
| HD1 | Gln | 70 | HG1 | 2.80 | |
| | Thr | 74 | OG1 | 2.83 | |
| | Thr | 74 | HG21 | 2.95 | |
| | Tyr | 71 | HA | 2.72 | |
| | Thr | 74 | HG1 | 2.72 | |
| | Lys | 5 | HZ3 | 3.48 | |
| | Tyr | 71 | CD1 | 3.10 | |
| | Tyr | 71 | HD1 | 2.81 | |
| | Tyr | 71 | CE1 | 3.45 | |
| | Tyr | 71 | HE1 | 3.45 | |
| HE1 | Thr | 74 | CA | 3.54 | |
| | Thr | 74 | CB | 2.24 | |
| | Thr | 74 | HB | 1.65 | |
| | Thr | 74 | OG1 | 2.13 | |
| | Thr | 74 | CG2 | 3.02 | |
| | Thr | 74 | HG21 | 2.87 | |
| | Thr | 74 | HG23 | 3.36 | |
| | Lys | 5 | HE1 | 1.73 | |
| | Tyr | 71 | HA | 3.58 | |
| | Thr | 74 | HG1 | 1.69 | |
| | Lys | 5 | CE | 2.59 | |
| | Lys | 5 | HE2 | 2.88 | |
| | Thr | 74 | O | 3.55 | |
| | Lys | 5 | HZ3 | 2.58 | |
| | Lys | 5 | NZ | 3.03 | |
| | Lys | 5 | HZ1 | 3.47 | |

TABLE 6
model05

| kobe2601 | H-RasT35S-GppNHp | | | | |
|---|---|---|---|---|---|
| atom | residue | number | atom | distance | angle |
| C11 | Tyr | 71 | HE1 | 3.44 | |
| F11 | Lys | 5 | HE2 | 3.53 | |
| | Lys | 5 | CE | 3.51 | |
| | Lys | 5 | HE1 | 2.59 | |
| | Leu | 56 | HD11 | 3.49 | |
| C10 | Thr | 74 | OG1 | 3.50 | |
| | Thr | 74 | HG21 | 3.21 | |
| | Tyr | 71 | CD1 | 3.08 | |
| | Tyr | 71 | HD1 | 2.60 | |
| | Tyr | 71 | CE1 | 3.07 | |
| | Tyr | 71 | HE1 | 2.58 | |
| | Leu | 56 | HD11 | 3.32 | |
| C9 | Gln | 70 | HB1 | 3.10 | |
| | Thr | 74 | HG21 | 3.49 | |
| | Tyr | 71 | CD1 | 3.20 | |
| | Tyr | 71 | HD1 | 3.17 | |
| | Tyr | 71 | CE1 | 2.88 | |
| | Tyr | 71 | HE1 | 2.56 | |
| | Glu | 37 | OE1 | 3.50 | |
| C8 | Tyr | 71 | HE1 | 3.40 | |
| | Glu | 37 | OE1 | 3.44 | |
| N8 | Gln | 70 | HG2 | 3.39 | |
| C7 | Met | 67 | HA | 3.29 | |
| | Gln | 70 | HB1 | 3.37 | |
| | Gln | 70 | HB2 | 3.58 | |
| | Gln | 70 | HG2 | 2.88 | |
| | Met | 67 | HB1 | 3.18 | |
| | Met | 67 | HE3 | 3.55 | |
| S7 | Met | 67 | CA | 3.46 | |
| | Met | 67 | HA | 2.93 | |
| | Met | 67 | C | 3.42 | |
| | Met | 67 | O | 2.66 | |
| | Gln | 70 | CB | 2.93 | |
| | Gln | 70 | HB1 | 2.35 | |
| | Gln | 70 | HB2 | 2.67 | |
| | Gln | 70 | HG2 | 3.17 | |
| | Tyr | 71 | HN | 3.33 | |
| | Tyr | 71 | CD1 | 3.47 | |
| | Tyr | 71 | CE1 | 3.12 | |
| | Tyr | 71 | HE1 | 3.51 | |
| | Tyr | 71 | CE2 | 3.35 | |
| | Tyr | 71 | CZ | 3.05 | |
| | Met | 67 | HB1 | 2.83 | |
| | Tyr | 71 | OH | 3.55 | |
| N7 | Met | 67 | HA | 2.82 | |
| | Met | 67 | CB | 3.52 | |
| | Gln | 70 | HG2 | 3.13 | |
| | Met | 67 | HB1 | 2.90 | |
| | Met | 67 | SD | 2.81 | |
| | Met | 67 | CE | 2.85 | |
| | Met | 67 | HE1 | 3.15 | |
| | Met | 67 | HE3 | 2.30 | |
| HAC | Met | 67 | CA | 2.71 | |
| | Met | 67 | HA | 1.96 | |
| | Met | 67 | CB | 2.69 | |
| | Gln | 70 | HB2 | 3.55 | |
| | Gln | 70 | HG2 | 3.33 | |
| | Met | 67 | HB1 | 2.30 | |
| | Met | 67 | CG | 2.91 | |
| | Met | 67 | HG2 | 3.27 | |
| | Met | 67 | SD | 2.36 | |
| | Met | 67 | CE | 2.36 | |
| | Met | 67 | HE1 | 2.98 | |
| | Met | 67 | HE2 | 3.20 | |
| | Met | 67 | HE3 | 1.73 | |
| N6 | Met | 67 | SD | 2.89 | |
| | Met | 67 | CE | 2.67 | |
| | Met | 67 | HE1 | 2.52 | |
| | Met | 67 | HE3 | 2.38 | |
| HAB | Met | 67 | SD | 2.66 | |
| | Met | 67 | CE | 2.82 | |
| | Met | 67 | HE1 | 2.52 | |
| | Met | 67 | HE3 | 2.91 | |
| C6 | Gln | 70 | HG2 | 3.52 | |
| | Met | 67 | CE | 3.10 | |
| | Met | 67 | HE1 | 2.69 | |
| | Met | 67 | HE3 | 2.68 | |
| C5 | Gln | 70 | CG | 3.42 | |
| | Gln | 70 | HG2 | 2.46 | |
| | Met | 67 | HE1 | 3.52 | |
| | Met | 67 | HE3 | 2.91 | |
| C4 | Gln | 70 | HG2 | 3.17 | |
| C1 | Met | 67 | HE1 | 2.93 | |
| N1 | Met | 67 | HE1 | 3.02 | |
| O1B | Met | 67 | SD | 3.57 | |
| | Met | 67 | HE1 | 2.88 | |
| H4 | Gln | 70 | HE22 | 3.17 | |
| | Gln | 70 | CG | 3.49 | |
| | Gln | 70 | HG2 | 3.05 | |
| | Gln | 70 | CD | 3.16 | |
| | Gln | 70 | NE2 | 2.81 | |
| | Gln | 70 | HE21 | 2.72 | |
| H5 | Met | 67 | HA | 3.54 | |
| | Gln | 70 | CB | 3.41 | |
| | Gln | 70 | HB2 | 3.39 | |
| | Gln | 70 | CG | 2.44 | |
| | Gln | 70 | HG2 | 1.42 | |
| | Gln | 70 | CD | 2.99 | |
| | Gln | 70 | OE1 | 3.54 | |
| | Gln | 70 | HG1 | 3.02 | |
| | Met | 67 | HE3 | 3.04 | |
| HD1 | Gln | 70 | CB | 3.20 | |
| | Gln | 70 | HB1 | 2.21 | |
| | Gln | 70 | HB2 | 3.46 | |
| | Gln | 70 | HG2 | 3.50 | |
| | Thr | 74 | HG21 | 3.54 | |
| | Tyr | 71 | HN | 3.42 | |
| | Tyr | 71 | CG | 3.56 | |
| | Tyr | 71 | CD1 | 2.83 | |
| | Tyr | 71 | HD1 | 3.02 | |
| | Tyr | 71 | CE1 | 2.70 | |
| | Tyr | 71 | HE1 | 2.79 | |
| | Tyr | 71 | CZ | 3.33 | |
| HE1 | Thr | 74 | OG1 | 2.85 | |
| | Thr | 74 | HG1 | 2.89 | |
| | Thr | 74 | HG21 | 3.33 | |
| | Leu | 56 | HD12 | 3.46 | |
| | Tyr | 71 | HA | 2.86 | |
| | Tyr | 71 | CG | 3.57 | |
| | Tyr | 71 | CD1 | 2.41 | |
| | Tyr | 71 | HD1 | 1.67 | |
| | Tyr | 71 | CE1 | 2.82 | |
| | Tyr | 71 | HE1 | 2.57 | |
| | Leu | 56 | CD1 | 3.38 | |
| | Leu | 56 | HD11 | 2.46 | |

TABLE 7
model06

| kobe2601 | H-RasT35S-GppNHp | | | | |
|---|---|---|---|---|---|
| atom | residue | number | atom | distance | angle |
| C11 | Tyr | 71 | HE1 | 3.44 | |
| F11 | Lys | 5 | HE2 | 3.53 | |
| | Lys | 5 | CE | 3.51 | |
| | Lys | 5 | HE1 | 2.59 | |
| | Leu | 56 | HD11 | 3.49 | |
| C10 | Thr | 74 | OG1 | 3.50 | |
| | Thr | 74 | HG21 | 3.21 | |
| | Tyr | 71 | CD1 | 3.08 | |
| | Tyr | 71 | HD1 | 2.60 | |
| | Tyr | 71 | CE1 | 3.07 | |
| | Tyr | 71 | HE1 | 2.58 | |
| | Leu | 56 | HD11 | 3.32 | |
| C9 | Gln | 70 | HB1 | 3.10 | |
| | Thr | 74 | HG21 | 3.49 | |
| | Tyr | 71 | CD1 | 3.20 | |
| | Tyr | 71 | HD1 | 3.17 | |
| | Tyr | 71 | CE1 | 2.88 | |
| | Tyr | 71 | HE1 | 2.56 | |
| | Glu | 37 | OE1 | 3.50 | |
| C8 | Tyr | 71 | HE1 | 3.40 | |
| | Glu | 37 | OE1 | 3.44 | |
| N8 | Gln | 70 | HG2 | 3.39 | |
| C7 | Met | 67 | HA | 3.29 | |
| | Gln | 70 | HB1 | 3.37 | |
| | Gln | 70 | HB2 | 3.58 | |
| | Gln | 70 | HG2 | 2.88 | |
| | Met | 67 | HB1 | 3.18 | |
| | Met | 67 | HE3 | 3.55 | |
| S7 | Met | 67 | CA | 3.46 | |
| | Met | 67 | HA | 2.93 | |
| | Met | 67 | C | 3.42 | |
| | Met | 67 | O | 2.66 | |
| | Gln | 70 | CB | 2.93 | |
| | Gln | 70 | HB1 | 2.35 | |
| | Gln | 70 | HB2 | 2.67 | |
| | Gln | 70 | HG2 | 3.17 | |
| | Tyr | 71 | HN | 3.33 | |
| | Tyr | 71 | CD1 | 3.47 | |
| | Tyr | 71 | CE1 | 3.12 | |
| | Tyr | 71 | HE1 | 3.51 | |
| | Tyr | 71 | CE2 | 3.35 | |
| | Tyr | 71 | CZ | 3.05 | |
| | Met | 67 | HB1 | 2.83 | |
| | Tyr | 71 | OH | 3.55 | |
| N7 | Met | 67 | HA | 2.82 | |
| | Met | 67 | CB | 3.52 | |
| | Gln | 70 | HG2 | 3.13 | |
| | Met | 67 | HB1 | 2.90 | |
| | Met | 67 | SD | 2.81 | |
| | Met | 67 | CE | 2.85 | |
| | Met | 67 | HE1 | 3.15 | |
| | Met | 67 | HE3 | 2.30 | |
| HAC | Met | 67 | CA | 2.71 | |
| | Met | 67 | HA | 1.96 | |
| | Met | 67 | CB | 2.69 | |
| | Gln | 70 | HB2 | 3.55 | |
| | Gln | 70 | HG2 | 3.33 | |
| | Met | 67 | HB1 | 2.30 | |
| | Met | 67 | CG | 2.91 | |
| | Met | 67 | HG2 | 3.27 | |
| | Met | 67 | SD | 2.36 | |
| | Met | 67 | CE | 2.36 | |
| | Met | 67 | HE1 | 2.98 | |
| | Met | 67 | HE2 | 3.20 | |
| | Met | 67 | HE3 | 1.73 | |
| N6 | Met | 67 | SD | 2.89 | |
| | Met | 67 | CE | 2.67 | |
| | Met | 67 | HE1 | 2.52 | |
| | Met | 67 | HE3 | 2.38 | |
| HAB | Met | 67 | SD | 2.66 | |
| | Met | 67 | CE | 2.82 | |
| | Met | 67 | HE1 | 2.52 | |
| | Met | 67 | HE3 | 2.91 | |
| C6 | Gln | 70 | HG2 | 3.52 | |
| | Met | 67 | CE | 3.10 | |
| | Met | 67 | HE1 | 2.69 | |
| | Met | 67 | HE3 | 2.68 | |
| C5 | Gln | 70 | CG | 3.42 | |
| | Gln | 70 | HG2 | 2.46 | |
| | Met | 67 | HE1 | 3.52 | |
| | Met | 67 | HE3 | 2.91 | |
| C4 | Gln | 70 | HG2 | 3.17 | |
| C1 | Met | 67 | HE1 | 2.93 | |
| N1 | Met | 67 | HE1 | 3.02 | |
| O1B | Met | 67 | SD | 3.57 | |
| | Met | 67 | HE1 | 2.88 | |
| H4 | Gln | 70 | HE22 | 3.17 | |
| | Gln | 70 | CG | 3.49 | |
| | Gln | 70 | HG2 | 3.05 | |
| | Gln | 70 | CD | 3.16 | |
| | Gln | 70 | NE2 | 2.81 | |
| | Gln | 70 | HE21 | 2.72 | |
| H5 | Met | 67 | HA | 3.54 | |
| | Gln | 70 | CB | 3.41 | |
| | Gln | 70 | HB2 | 3.39 | |
| | Gln | 70 | CG | 2.44 | |
| | Gln | 70 | HG2 | 1.42 | |
| | Gln | 70 | CD | 2.99 | |
| | Gln | 70 | OE1 | 3.54 | |
| | Gln | 70 | HG1 | 3.02 | |
| | Met | 67 | HE3 | 3.04 | |
| HD1 | Gln | 70 | CB | 3.20 | |
| | Gln | 70 | HB1 | 2.21 | |
| | Gln | 70 | HB2 | 3.46 | |
| | Gln | 70 | HG2 | 3.50 | |
| | Thr | 74 | HG21 | 3.54 | |
| | Tyr | 71 | HN | 3.42 | |
| | Tyr | 71 | CG | 3.56 | |
| | Tyr | 71 | CD1 | 2.83 | |
| | Tyr | 71 | HD1 | 3.02 | |
| | Tyr | 71 | CE1 | 2.70 | |
| | Tyr | 71 | HE1 | 2.79 | |
| | Tyr | 71 | CZ | 3.33 | |
| HE1 | Thr | 74 | OG1 | 2.85 | |
| | Thr | 74 | HG1 | 2.89 | |
| | Thr | 74 | HG21 | 3.33 | |
| | Leu | 56 | HD12 | 3.46 | |
| | Tyr | 71 | HA | 2.86 | |
| | Tyr | 71 | CG | 3.57 | |
| | Tyr | 71 | CD1 | 2.41 | |
| | Tyr | 71 | HD1 | 1.67 | |
| | Tyr | 71 | CE1 | 2.82 | |
| | Tyr | 71 | HE1 | 2.57 | |
| | Leu | 56 | CD1 | 3.38 | |
| | Leu | 56 | HD11 | 2.46 | |

TABLE 8
model07

| kobe2601 | H-RasT35S-GppNHp | | | | |
|---|---|---|---|---|---|
| atom | residue | number | atom | distance | angle |
| C13 | Thr | 74 | HG1 | 2.83 | |
| | Thr | 74 | CG2 | 3.28 | |
| | Thr | 74 | HG21 | 2.97 | |
| | Thr | 74 | HB | 3.33 | |
| | Thr | 74 | HG23 | 2.87 | |
| | Tyr | 71 | HD1 | 3.35 | |
| | Tyr | 71 | HE1 | 3.45 | |
| C12 | Thr | 74 | CB | 3.47 | |
| | Thr | 74 | OG1 | 3.36 | |
| | Thr | 74 | HG1 | 2.62 | |
| | Thr | 74 | HB | 2.89 | |
| | Thr | 74 | HG23 | 3.38 | |
| | Lys | 5 | HZ3 | 3.05 | |
| | Tyr | 71 | HD1 | 3.49 | |
| | Lys | 5 | HE1 | 3.31 | |
| C11 | Thr | 74 | HG1 | 3.49 | |
| | Thr | 74 | HB | 2.77 | |
| | Thr | 74 | HG23 | 3.54 | |
| | Lys | 5 | HE1 | 3.53 | |
| | Lys | 5 | HE2 | 3.51 | |
| F11 | Thr | 74 | HB | 3.25 | |
| | Lys | 5 | CE | 3.34 | |
| | Lys | 5 | HE1 | 3.19 | |
| | Lys | 5 | HE2 | 2.60 | |
| C10 | Thr | 74 | HB | 3.14 | |
| | Thr | 74 | HG23 | 3.24 | |
| C9 | Thr | 74 | HB | 3.56 | |
| | Thr | 74 | HG23 | 2.71 | |
| C8 | Thr | 74 | CG2 | 3.28 | |
| | Thr | 74 | HG21 | 3.21 | |
| | Thr | 74 | HG23 | 2.49 | |
| N8 | Gln | 70 | CG | 3.47 | |
| | Gln | 70 | HG1 | 3.20 | |
| | Gln | 70 | HG2 | 3.07 | |
| | Thr | 74 | HG21 | 3.41 | |
| | Thr | 74 | HG23 | 2.95 | |
| HAD | Gln | 70 | CG | 2.90 | |
| | Gln | 70 | HG1 | 2.68 | |
| | Gln | 70 | HG2 | 2.33 | |
| | Gln | 70 | CD | 3.47 | |
| | Thr | 74 | HG21 | 3.34 | |
| | Thr | 74 | HG23 | 3.40 | |
| C7 | Gln | 70 | OE1 | 3.32 | |
| | Gln | 70 | HG2 | 3.44 | |
| | Thr | 74 | HG23 | 3.51 | |
| N7 | Gln | 70 | OE1 | 2.70 | *** |
| | Gln | 70 | HG2 | 3.03 | |
| | Gln | 70 | CD | 3.35 | |
| HAC | Gln | 70 | OE1 | 2.87 | |
| N6 | Gln | 70 | OE1 | 2.64 | *** |
| | Gln | 70 | CG | 2.99 | |
| | Gln | 70 | HG1 | 3.52 | |
| | Gln | 70 | HG2 | 2.13 | |
| | Gln | 70 | CD | 3.12 | |
| HAB | Gln | 70 | OE1 | 2.02 | |
| | Gln | 70 | CB | 3.38 | |
| | Gln | 70 | HB2 | 3.29 | |
| | Gln | 70 | CG | 2.41 | |
| | Gln | 70 | HG1 | 3.19 | |
| | Gln | 70 | HG2 | 1.62 | |
| | Gln | 70 | CD | 2.51 | |
| C6 | Gln | 70 | HG2 | 2.60 | |
| C5 | Gln | 70 | CG | 3.49 | |
| | Gln | 70 | HG2 | 2.45 | |
| | Met | 67 | HB1 | 3.60 | |
| | Met | 67 | HE2 | 3.36 | |
| | Met | 67 | HE3 | 3.35 | |
| C4 | Met | 67 | HB1 | 3.37 | |
| | Met | 67 | CE | 2.86 | |
| | Met | 67 | HE1 | 3.44 | |
| | Met | 67 | HE2 | 2.40 | |
| | Met | 67 | HE3 | 2.43 | |
| C3 | Met | 67 | CE | 3.49 | |
| | Met | 67 | HE2 | 2.87 | |
| | Met | 67 | HE3 | 3.33 | |
| N3 | Met | 67 | CE | 3.37 | |
| | Met | 67 | HE1 | 3.22 | |
| | Met | 67 | HE2 | 2.93 | |
| | Met | 67 | HE3 | 3.40 | |
| | Glu | 63 | OE2 | 3.45 | * |
| | Tyr | 71 | OH | 3.52 | * |
| O3B | Glu | 37 | HB1 | 3.54 | |
| O3A | Glu | 63 | CD | 3.05 | |
| | Met | 67 | CE | 2.53 | |
| | Met | 67 | HE1 | 2.08 | |
| | Met | 67 | HE2 | 2.54 | |
| | Met | 67 | HE3 | 2.59 | |
| | Glu | 63 | HB1 | 3.28 | |
| | Glu | 63 | OE2 | 2.44 | *** |
| | Tyr | 71 | CZ | 3.58 | |
| | Tyr | 71 | OH | 2.53 | *** |
| | Tyr | 71 | HH | 2.84 | |
| | Glu | 63 | OE1 | 3.35 | * |
| H4 | Met | 67 | CB | 3.47 | |
| | Met | 67 | HB1 | 2.54 | |
| | Met | 67 | HG2 | 3.48 | |
| | Met | 67 | CE | 2.21 | |
| | Met | 67 | HE1 | 2.77 | |
| | Met | 67 | HE2 | 2.18 | |
| | Met | 67 | HE3 | 1.50 | |
| | Tyr | 71 | CZ | 3.53 | |
| | Tyr | 71 | OH | 3.33 | |
| H5 | Gln | 70 | CB | 3.15 | |
| | Gln | 70 | HB1 | 2.73 | |
| | Gln | 70 | HB2 | 3.34 | |
| | Gln | 70 | CG | 2.81 | |
| | Gln | 70 | HG1 | 3.31 | |
| | Gln | 70 | HG2 | 1.86 | |
| | Met | 67 | HB1 | 3.04 | |
| | Met | 67 | HG2 | 3.38 | |
| | Met | 67 | HE3 | 3.41 | |
| HD1 | Thr | 74 | HG23 | 3.38 | |
| HE1 | Thr | 74 | HB | 3.59 | |
| HE2 | Thr | 74 | OG1 | 3.10 | |
| | Thr | 74 | HG1 | 2.31 | |
| | Thr | 74 | HB | 3.17 | |
| | Lys | 5 | NZ | 2.82 | |
| | Lys | 5 | HZ2 | 3.52 | |
| | Lys | 5 | HZ3 | 1.97 | |
| | Leu | 56 | HD12 | 3.36 | |
| | Tyr | 71 | HD1 | 2.90 | |
| | Tyr | 71 | HE1 | 3.43 | |
| | Lys | 5 | CE | 3.00 | |
| | Lys | 5 | HE1 | 2.67 | |
| | Lys | 5 | HE2 | 3.02 | |
| | Lys | 5 | HZ1 | 3.42 | |
| HD2 | Gln | 70 | HG1 | 3.41 | |
| | Thr | 74 | HG1 | 2.97 | |
| | Thr | 74 | HG21 | 3.10 | |
| | Thr | 74 | HG23 | 3.52 | |
| | Tyr | 71 | CD1 | 3.24 | |
| | Tyr | 71 | HD1 | 2.74 | |
| | Tyr | 71 | CE1 | 3.15 | |
| | Tyr | 71 | HE1 | 2.53 | |

TABLE 9
model08

| kobe2601 | H-RasT35S-GppNHp | | | | |
|---|---|---|---|---|---|
| atom | residue | number | atom | distance | angle |
| C13 | Gln | 70 | HG2 | 3.46 | |
| C11 | Lys | 5 | HE1 | 3.38 | |
| F11 | Thr | 74 | HB | 3.54 | |
| | Lys | 5 | CE | 3.08 | |
| | Lys | 5 | HE1 | 2.75 | |
| | Lys | 5 | HE2 | 2.75 | |
| | Lys | 5 | NZ | 3.40 | |
| | Lys | 5 | HZ3 | 2.78 | |
| C10 | Leu | 56 | HD21 | 3.40 | |
| | Tyr | 71 | HA | 3.52 | |
| | Tyr | 71 | CD1 | 3.38 | |
| | Tyr | 71 | HD1 | 3.13 | |
| | Tyr | 71 | CE1 | 3.50 | |
| | Tyr | 71 | HE1 | 3.36 | |
| | Thr | 74 | OG1 | 3.59 | |
| | Lys | 5 | HE1 | 3.20 | |
| C9 | Gln | 70 | HG1 | 3.59 | |
| | Tyr | 71 | HA | 3.56 | |
| | Tyr | 71 | CD1 | 2.98 | |
| | Tyr | 71 | HD1 | 3.20 | |
| | Tyr | 71 | CE1 | 2.85 | |
| | Tyr | 71 | HE1 | 2.98 | |
| | Tyr | 71 | CZ | 3.41 | |
| C8 | Gln | 70 | HG1 | 3.48 | |
| | Gln | 70 | HG2 | 3.19 | |
| N8 | Gln | 70 | HG2 | 3.00 | |
| HAD | Met | 67 | HB1 | 2.82 | |
| | Gln | 70 | HB1 | 3.42 | |
| | Gln | 70 | HG2 | 3.40 | |
| | Tyr | 71 | CE1 | 3.43 | |
| | Tyr | 71 | CE2 | 3.00 | |
| | Tyr | 71 | HE2 | 3.23 | |
| | Tyr | 71 | CZ | 2.89 | |
| | Tyr | 71 | OH | 3.17 | |
| C7 | Gln | 70 | HG2 | 3.07 | |
| S7 | Gln | 70 | HG2 | 3.51 | |
| N7 | Met | 67 | HB1 | 3.18 | |
| | Met | 67 | HE3 | 2.93 | |
| | Met | 67 | SD | 2.87 | |
| | Met | 67 | CE | 3.34 | |
| HAC | Met | 67 | CA | 3.55 | |
| | Met | 67 | CB | 2.99 | |
| | Met | 67 | HA | 3.03 | |
| | Met | 67 | HB1 | 2.20 | |
| | Met | 67 | HE3 | 2.76 | |
| | Met | 67 | CG | 3.18 | |
| | Met | 67 | SD | 2.26 | |
| | Met | 67 | CE | 3.06 | |
| N6 | Met | 67 | HE3 | 2.56 | |
| | Met | 67 | SD | 2.87 | |
| | Met | 67 | CE | 2.88 | |
| | Met | 67 | HE1 | 2.85 | |
| HAB | Met | 67 | HE3 | 2.95 | |
| | Met | 67 | SD | 2.71 | |
| | Met | 67 | CE | 2.90 | |
| | Met | 67 | HE1 | 2.63 | |
| C6 | Met | 67 | HE3 | 2.50 | |
| | Met | 67 | CE | 3.11 | |
| | Met | 67 | HE1 | 2.97 | |
| C5 | Met | 67 | HE3 | 2.91 | |
| | Gln | 70 | HG2 | 2.89 | |
| C4 | Gln | 70 | HG2 | 3.38 | |
| C1 | Met | 67 | HE3 | 3.07 | |
| | Met | 67 | CE | 3.45 | |
| | Met | 67 | HE1 | 2.90 | |
| N1 | Met | 67 | HE3 | 3.58 | |
| | Met | 67 | CE | 3.56 | |
| | Met | 67 | HE1 | 2.70 | |
| O1B | Met | 67 | SD | 3.59 | |
| | Met | 67 | CE | 3.36 | |
| | Met | 67 | HE1 | 2.58 | |
| O1A | Met | 67 | HE1 | 3.57 | |
| H4 | Gln | 70 | HB2 | 3.27 | |
| | Gln | 70 | CG | 3.46 | |
| | Gln | 70 | HG2 | 3.04 | |
| | Gln | 70 | CD | 3.07 | |
| | Gln | 70 | NE2 | 2.89 | |
| | Gln | 70 | HE21 | 2.96 | |
| | Gln | 70 | HE22 | 3.21 | |
| H5 | Met | 67 | HE3 | 3.22 | |
| | Gln | 70 | CB | 3.23 | |
| | Gln | 70 | HB1 | 3.13 | |
| | Gln | 70 | HB2 | 3.06 | |
| | Gln | 70 | CG | 2.87 | |
| | Gln | 70 | HG2 | 1.93 | |
| | Gln | 70 | CD | 3.59 | |
| | Gln | 70 | HE21 | 3.28 | |
| HD1 | Tyr | 71 | HA | 3.12 | |
| | Tyr | 71 | CG | 2.61 | |
| | Tyr | 71 | CD1 | 2.09 | |
| | Tyr | 71 | HD1 | 2.58 | |
| | Tyr | 71 | CD2 | 3.01 | |
| | Tyr | 71 | CE1 | 2.08 | |
| | Tyr | 71 | HE1 | 2.57 | |
| | Tyr | 71 | CE2 | 3.01 | |
| | Tyr | 71 | CZ | 2.58 | |
| | Tyr | 71 | OH | 3.51 | |
| HE1 | Thr | 74 | HG1 | 3.45 | |
| | Leu | 56 | HG | 2.76 | |
| | Leu | 56 | HD21 | 2.71 | |
| | Tyr | 71 | HA | 3.22 | |
| | Tyr | 71 | CD1 | 2.95 | |
| | Tyr | 71 | HD1 | 2.40 | |
| | Tyr | 71 | CE1 | 3.33 | |
| | Tyr | 71 | HE1 | 3.16 | |
| | Thr | 74 | OG1 | 3.49 | |
| | Lys | 5 | CE | 3.43 | |
| | Lys | 5 | HE1 | 2.45 | |
| | Leu | 56 | CG | 3.60 | |
| | Leu | 56 | CD2 | 3.53 | |
| HD2 | Gln | 70 | HG2 | 3.31 | |
| | Gln | 70 | HE21 | 3.25 | |

TABLE 10
model09

| kobe2601 | H-RasT35S-GppNHp | | | | |
|---|---|---|---|---|---|
| atom | residue | number | atom | distance | angle |
| C13 | Tyr | 71 | HE1 | 2.78 | |
| | Tyr | 71 | CZ | 3.16 | |
| | Tyr | 71 | CE1 | 2.95 | |
| | Tyr | 71 | OH | 3.23 | |
| | Tyr | 71 | HH | 3.33 | |
| C12 | Tyr | 71 | CZ | 3.53 | |
| | Leu | 56 | CD1 | 2.96 | |
| | Leu | 56 | HD11 | 2.89 | |
| | Leu | 56 | HD12 | 2.75 | |
| | Leu | 56 | HD13 | 2.75 | |
| | Tyr | 71 | CE1 | 3.40 | |
| C11 | Lys | 5 | HE1 | 3.44 | |
| | Leu | 56 | CD1 | 3.48 | |
| | Leu | 56 | HD12 | 3.08 | |
| | Leu | 56 | HD13 | 3.04 | |
| | Lys | 5 | HZ3 | 3.47 | |
| F11 | Lys | 5 | HD1 | 3.53 | |
| | Lys | 5 | CE | 3.51 | |
| | Lys | 5 | HE1 | 2.83 | |
| | Leu | 56 | HB2 | 3.28 | |
| | Leu | 56 | CD1 | 3.10 | |
| | Leu | 56 | HD12 | 2.64 | |
| | Leu | 56 | HD13 | 2.62 | |
| | Lys | 5 | NZ | 3.38 | |
| | Lys | 5 | HZ2 | 3.03 | |
| | Lys | 5 | HZ3 | 3.14 | |
| C10 | Lys | 5 | HZ3 | 3.06 | |
| C8 | Tyr | 71 | HE1 | 3.32 | |
| N8 | Tyr | 71 | HE1 | 3.36 | |
| HAD | Tyr | 71 | HE1 | 2.96 | |
| | Tyr | 71 | OH | 3.58 | |
| C6 | Met | 67 | HE1 | 3.55 | |
| C4 | Gln | 70 | HG2 | 2.76 | |
| C3 | Gln | 70 | HG2 | 3.27 | |
| | Met | 67 | HG2 | 3.00 | |
| | Met | 67 | HE1 | 3.16 | |
| N3 | Gln | 70 | HG2 | 3.03 | |
| | Met | 67 | HG2 | 2.69 | |
| O3B | Met | 67 | HA | 3.13 | |
| | Met | 67 | CG | 2.95 | |
| | Met | 67 | HG1 | 3.10 | |
| | Met | 67 | HG2 | 1.99 | |
| O3A | Gln | 70 | OE1 | 2.38 | *** |
| | Gln | 70 | CB | 3.30 | |
| | Gln | 70 | HB2 | 2.88 | |
| | Gln | 70 | CG | 2.79 | |
| | Gln | 70 | CD | 2.89 | |
| | Gln | 70 | HG2 | 2.12 | |
| C2 | Met | 67 | CG | 3.59 | |
| | Met | 67 | HG2 | 2.77 | |
| | Met | 67 | SD | 3.54 | |
| | Met | 67 | CE | 3.19 | |
| | Met | 67 | HE1 | 2.53 | |
| | Met | 67 | HE3 | 3.26 | |
| C1 | Met | 67 | CE | 3.25 | |
| | Met | 67 | HE1 | 2.77 | |
| | Met | 67 | HE3 | 2.88 | |
| N1 | Glu | 63 | OE2 | 3.57 | * |
| | Met | 67 | CE | 3.13 | |
| | Met | 67 | HE1 | 3.10 | |
| | Met | 67 | HE3 | 2.41 | |
| O1B | Met | 67 | HE3 | 3.06 | |
| O1A | Glu | 63 | OE2 | 2.84 | *** |
| | Met | 67 | SD | 2.92 | |
| | Met | 67 | CE | 2.94 | |
| | Met | 67 | HE1 | 3.24 | |
| | Met | 67 | HE3 | 2.36 | |
| H2 | Met | 67 | CG | 2.94 | |
| | Met | 67 | HG1 | 3.47 | |
| | Met | 67 | HG2 | 2.25 | |
| | Met | 67 | SD | 2.85 | |
| | Met | 67 | CE | 3.06 | |
| | Met | 67 | HE1 | 2.66 | |
| | Met | 67 | HE3 | 3.23 | |
| H4 | Gln | 70 | CG | 2.99 | |
| | Gln | 70 | HG1 | 3.38 | |
| | Gln | 70 | HG2 | 1.93 | |
| HE1 | Lys | 5 | NZ | 3.19 | |
| | Lys | 5 | HZ2 | 3.26 | |
| | Lys | 5 | HZ3 | 2.30 | |
| HE2 | Tyr | 71 | CG | 3.59 | |
| | Tyr | 71 | CD2 | 3.46 | |
| | Tyr | 71 | CE2 | 3.23 | |
| | Tyr | 71 | CZ | 3.11 | |
| | Leu | 56 | CG | 3.52 | |
| | Leu | 56 | CD1 | 2.01 | |
| | Leu | 56 | HD11 | 1.84 | |
| | Leu | 56 | HD12 | 1.91 | |
| | Leu | 56 | HD13 | 2.08 | |
| | Tyr | 71 | CD1 | 3.47 | |
| | Tyr | 71 | CE1 | 3.24 | |
| | Tyr | 71 | HH | 3.54 | |
| HD2 | Tyr | 71 | HE1 | 2.28 | |
| | Tyr | 71 | CE2 | 3.40 | |
| | Tyr | 71 | CZ | 2.34 | |
| | Tyr | 71 | CD1 | 3.49 | |
| | Tyr | 71 | CE1 | 2.40 | |
| | Tyr | 71 | OH | 2.18 | |
| | Tyr | 71 | HH | 2.35 | |

TABLE 11

| | kobe2601 | H-RasT35S-GppNHp | | | | |
|---|---|---|---|---|---|---|
| model10 | atom | residue | number | atom | distance | angle |
| | C13 | Gln | 70 | HG1 | 3.40 | |
| | | Gln | 70 | HG2 | 3.50 | |
| | | Thr | 74 | HG21 | 3.05 | |
| | | Tyr | 71 | HD2 | 3.28 | |
| | | Tyr | 71 | HE2 | 2.90 | |
| | | Thr | 74 | HG1 | 3.58 | |
| | C12 | Thr | 74 | HB | 3.46 | |
| | | Thr | 74 | HG21 | 3.26 | |
| | | Lys | 5 | HE1 | 3.38 | |
| | | Thr | 74 | HG1 | 3.09 | |
| | C11 | Lys | 5 | HE1 | 3.27 | |
| | F11 | Lys | 5 | HE2 | 3.55 | |
| | | Lys | 5 | CE | 3.17 | |
| | | Lys | 5 | HE1 | 2.62 | |
| | | Lys | 5 | NZ | 2.89 | |
| | | Lys | 5 | HZ1 | 2.60 | |
| | | Lys | 5 | HZ3 | 2.59 | |
| | C10 | Tyr | 71 | HE2 | 3.30 | |
| | | Ser | 39 | HB1 | 3.08 | |
| | | Ser | 39 | HG | 3.51 | |
| | C9 | Tyr | 71 | CE2 | 3.50 | |
| | | Tyr | 71 | HE2 | 2.43 | |
| | C8 | Tyr | 71 | HD2 | 3.59 | |
| | | Tyr | 71 | CE2 | 3.09 | |
| | | Tyr | 71 | HE2 | 2.15 | |
| | N8 | Gln | 70 | HG2 | 3.44 | |
| | | Tyr | 71 | CE2 | 2.95 | |
| | | Tyr | 71 | HE2 | 2.27 | |
| | | Tyr | 71 | CZ | 3.56 | |
| | | Tyr | 71 | OH | 3.52 | * |
| | HAD | Gln | 70 | CG | 3.38 | |
| | | Gln | 70 | HG1 | 3.40 | |
| | | Gln | 70 | HG2 | 2.50 | |
| | | Tyr | 71 | CE2 | 3.21 | |
| | | Tyr | 71 | HE2 | 2.80 | |
| | C7 | Tyr | 71 | CE2 | 3.26 | |
| | | Tyr | 71 | HE2 | 2.65 | |
| | | Tyr | 71 | CZ | 3.39 | |
| | | Met | 67 | HE3 | 3.53 | |
| | | Tyr | 71 | OH | 2.85 | |
| | S7 | Tyr | 71 | HE2 | 3.11 | |
| | | Tyr | 71 | OH | 3.07 | |
| | | Tyr | 71 | HH | 3.58 | |
| | | Asp | 38 | CB | 3.60 | |
| | | Asp | 38 | HB2 | 2.56 | |
| | N7 | Met | 67 | HB1 | 3.30 | |
| | | Tyr | 71 | HE2 | 3.58 | |
| | | Met | 67 | CE | 2.95 | |
| | | Met | 67 | HE1 | 3.45 | |
| | | Met | 67 | HE2 | 2.83 | |
| | | Met | 67 | HE3 | 2.23 | |
| | | Tyr | 71 | OH | 2.94 | *** |
| | HAC | Met | 67 | CE | 2.40 | |
| | | Met | 67 | HE1 | 2.65 | |
| | | Met | 67 | HE2 | 2.50 | |
| | | Met | 67 | HE3 | 1.75 | |
| | | Tyr | 71 | OH | 3.02 | |
| | N6 | Met | 67 | CB | 3.14 | |
| | | Met | 67 | HB1 | 2.25 | |
| | | Met | 67 | HB2 | 3.39 | |
| | | Met | 67 | CG | 3.53 | |
| | | Met | 67 | HG2 | 3.16 | |
| | | Met | 67 | CE | 2.84 | |
| | | Met | 67 | HE2 | 2.59 | |
| | | Met | 67 | HE3 | 2.21 | |
| | | Tyr | 71 | OH | 3.60 | * |
| | HAB | Met | 67 | CA | 3.56 | |
| | | Met | 67 | CB | 2.43 | |
| | | Met | 67 | HB1 | 1.49 | |
| | | Met | 67 | HB2 | 2.65 | |
| | | Met | 67 | CG | 3.21 | |
| | | Met | 67 | HG2 | 3.08 | |
| | | Gln | 70 | HB1 | 3.37 | |
| | | Tyr | 71 | CZ | 3.48 | |
| | | Met | 67 | CE | 3.11 | |
| | | Met | 67 | HE2 | 3.11 | |
| | | Met | 67 | HE3 | 2.34 | |
| | | Tyr | 71 | OH | 3.41 | |
| | C6 | Met | 67 | CB | 3.51 | |
| | | Met | 67 | HB1 | 2.67 | |
| | | Met | 67 | CG | 3.49 | |
| | | Met | 67 | HG2 | 2.75 | |
| | | Gln | 70 | HG2 | 3.36 | |
| | | Met | 67 | CE | 3.17 | |
| | | Met | 67 | HE2 | 2.48 | |
| | | Met | 67 | HE3 | 2.97 | |
| | C5 | Met | 67 | HA | 3.57 | |
| | | Met | 67 | CB | 3.24 | |
| | | Met | 67 | HB1 | 2.46 | |
| | | Met | 67 | CG | 3.23 | |
| | | Met | 67 | HG2 | 2.39 | |
| | | Gln | 70 | HB1 | 3.36 | |
| | | Gln | 70 | HB2 | 3.43 | |
| | | Gln | 70 | HG2 | 2.76 | |
| | | Gln | 70 | HE21 | 3.60 | |
| | | Met | 67 | HE2 | 3.33 | |
| | C4 | Met | 67 | HG2 | 3.08 | |
| | | Gln | 70 | HG2 | 3.15 | |
| | | Gln | 70 | HE21 | 3.12 | |
| | C3 | Gln | 70 | HE21 | 3.37 | |
| | C1 | Met | 67 | CE | 3.60 | |
| | | Met | 67 | HE2 | 2.67 | |
| | N1 | Met | 67 | CE | 3.53 | |
| | | Met | 67 | HE2 | 2.73 | |
| | | Met | 67 | HE3 | 3.58 | |
| | O1B | Met | 67 | CE | 2.95 | |
| | | Met | 67 | HE1 | 3.04 | |
| | | Met | 67 | HE2 | 2.57 | |
| | | Met | 67 | HE3 | 2.75 | |
| | H4 | Met | 67 | HG2 | 3.52 | |
| | | Gln | 70 | HB2 | 3.13 | |
| | | Gln | 70 | HG2 | 3.33 | |
| | | Gln | 70 | NE2 | 3.50 | |
| | | Gln | 70 | HE21 | 3.15 | |
| | H5 | Met | 67 | CA | 3.23 | |
| | | Met | 67 | HA | 2.78 | |
| | | Met | 67 | CB | 2.78 | |
| | | Met | 67 | HB1 | 1.95 | |
| | | Met | 67 | CG | 3.21 | |
| | | Met | 67 | HG2 | 2.64 | |
| | | Met | 67 | O | 3.54 | |
| | | Gln | 70 | CB | 2.76 | |
| | | Gln | 70 | HB1 | 2.33 | |
| | | Gln | 70 | HB2 | 2.59 | |
| | | Gln | 70 | CG | 3.02 | |
| | | Gln | 70 | HG2 | 2.37 | |
| | HD1 | Tyr | 71 | HE2 | 2.81 | |
| | | Asp | 38 | HB2 | 3.06 | |
| | | Asp | 38 | OD1 | 3.43 | |
| | HE1 | Asp | 38 | O | 3.55 | |
| | | Ser | 39 | CB | 2.97 | |
| | | Ser | 39 | HB1 | 2.03 | |
| | | Ser | 39 | OG | 3.21 | |
| | | Ser | 39 | HG | 2.58 | |
| | | Ser | 39 | HB2 | 3.49 | |
| | HE2 | Thr | 74 | CG2 | 3.26 | |
| | | Lys | 5 | HE2 | 3.52 | |
| | | Thr | 74 | CB | 2.98 | |
| | | Thr | 74 | HB | 2.39 | |
| | | Thr | 74 | OG1 | 2.94 | |
| | | Thr | 74 | HG21 | 2.72 | |
| | | Lys | 5 | HE1 | 2.81 | |
| | | Thr | 74 | HG1 | 2.36 | |
| | HD2 | Thr | 74 | CG2 | 3.32 | |
| | | Gln | 70 | CG | 2.90 | |
| | | Gln | 70 | HG1 | 2.43 | |
| | | Gln | 70 | HG2 | 2.52 | |
| | | Gln | 70 | HE21 | 3.26 | |
| | | Thr | 74 | HG21 | 2.26 | |
| | | Tyr | 71 | HD2 | 3.45 | |
| | | Tyr | 71 | HE2 | 3.45 | |
| | | Thr | 74 | HG1 | 3.57 | |

TABLE 12

| | kobe2601 | H-RasT35S-GppNHp | | | | |
|---|---|---|---|---|---|---|
| model11 | atom | residue | number | atom | distance | angle |
| | C13 | Tyr | 71 | CE2 | 3.29 | |
| | | Leu | 56 | HD12 | 3.45 | |
| | | Tyr | 71 | HE2 | 2.47 | |
| | | Asp | 38 | HB1 | 3.30 | |
| | C12 | Leu | 56 | CD1 | 3.51 | |
| | | Leu | 56 | HD12 | 2.85 | |
| | | Leu | 56 | HD13 | 3.20 | |
| | | Tyr | 71 | HE2 | 3.30 | |
| | | Asp | 38 | HB1 | 3.51 | |
| | F11 | Lys | 5 | HE1 | 2.56 | |
| | | Leu | 56 | HD13 | 3.55 | |
| | | Leu | 56 | HD22 | 3.35 | |
| | | Thr | 74 | HG1 | 3.43 | |
| | | Lys | 5 | CE | 3.21 | |
| | | Lys | 5 | NZ | 2.94 | |
| | | Lys | 5 | HZ2 | 2.60 | |
| | | Lys | 5 | HZ3 | 2.68 | |
| | C9 | Tyr | 71 | HE2 | 3.22 | |
| | C8 | Tyr | 71 | CE2 | 3.46 | |
| | | Tyr | 71 | HE2 | 2.42 | |
| | N8 | Tyr | 71 | HE2 | 2.57 | |
| | HAD | Tyr | 71 | CE2 | 3.58 | |
| | | Tyr | 71 | HE2 | 2.64 | |
| | | Tyr | 71 | OH | 3.37 | |
| | | Tyr | 71 | HH | 3.25 | |
| | C7 | Tyr | 71 | HE2 | 3.43 | |
| | S7 | Gln | 70 | HG2 | 3.47 | |
| | HAC | Met | 67 | HE2 | 3.55 | |
| | N6 | Met | 67 | HE1 | 3.23 | |
| | C6 | Met | 67 | CE | 3.37 | |
| | | Met | 67 | HE1 | 2.45 | |
| | | Met | 67 | HE2 | 3.45 | |
| | C5 | Met | 67 | CE | 3.37 | |
| | | Met | 67 | HE1 | 2.62 | |
| | | Gln | 70 | HG2 | 3.20 | |
| | | Gln | 70 | OE1 | 3.55 | |
| | | Met | 67 | HE2 | 3.51 | |
| | C4 | Met | 67 | HA | 3.14 | |
| | | Met | 67 | SD | 3.45 | |
| | | Met | 67 | CE | 3.55 | |
| | | Met | 67 | HE1 | 2.82 | |
| | | Gln | 70 | HB1 | 3.14 | |
| | | Gln | 70 | HB2 | 3.12 | |
| | | Gln | 70 | CB | 3.40 | |
| | | Gln | 70 | CG | 3.36 | |
| | | Gln | 70 | HG2 | 3.00 | |
| | | Gln | 70 | CD | 3.27 | |
| | | Gln | 70 | OE1 | 3.11 | |
| | C3 | Met | 67 | HE1 | 2.86 | |
| | | Gln | 70 | HB2 | 3.57 | |
| | | Gln | 70 | CD | 3.51 | |
| | | Gln | 70 | OE1 | 3.21 | |
| | N3 | Gln | 70 | HB2 | 3.20 | |
| | | Gln | 70 | CD | 3.58 | |
| | O3A | Gln | 70 | HE22 | 3.56 | |
| | | Gln | 70 | NE2 | 3.29 | *** |
| | | Gln | 70 | HE21 | 3.38 | |
| | | Ala | 66 | O | 3.32 | * |
| | | Met | 67 | HA | 3.37 | |
| | | Gln | 70 | HB1 | 3.42 | |
| | | Gln | 70 | HB2 | 2.15 | |
| | | Gln | 70 | CB | 3.15 | |
| | | Gln | 70 | CD | 3.44 | |
| | C2 | Met | 67 | HE1 | 2.70 | |
| | C1 | Met | 67 | CE | 3.56 | |
| | | Met | 67 | HE1 | 2.49 | |
| | N1 | Met | 67 | HE1 | 3.29 | |
| | H2 | Met | 67 | HE1 | 3.26 | |
| | H4 | Met | 67 | CA | 3.37 | |
| | | Met | 67 | HA | 2.37 | |
| | | Met | 67 | HB1 | 3.39 | |
| | | Met | 67 | SD | 3.53 | |
| | | Met | 67 | HE1 | 3.59 | |
| | | Gln | 70 | HB1 | 2.13 | |
| | | Gln | 70 | HB2 | 2.25 | |
| | | Gln | 70 | CB | 2.47 | |
| | | Gln | 70 | CG | 2.80 | |
| | | Gln | 70 | HG2 | 2.58 | |
| | | Gln | 70 | CD | 3.07 | |
| | | Gln | 70 | OE1 | 3.31 | |
| | H5 | Met | 67 | HB1 | 3.11 | |
| | | Met | 67 | HE1 | 3.43 | |
| | | Gln | 70 | HB1 | 3.57 | |
| | | Gln | 70 | HG2 | 2.84 | |
| | | Tyr | 71 | HE2 | 3.45 | |
| | HD1 | Gln | 70 | HG2 | 3.37 | |
| | HE1 | Thr | 74 | CG2 | 3.57 | |
| | | Thr | 74 | HG21 | 3.15 | |
| | | Thr | 74 | HG23 | 3.34 | |
| | | Thr | 74 | HB | 3.17 | |
| | | Thr | 74 | HG1 | 3.36 | |
| | | Lys | 5 | HZ3 | 3.42 | |
| | HE2 | Leu | 56 | CD1 | 2.53 | |
| | | Leu | 56 | HD11 | 3.17 | |
| | | Leu | 56 | HD12 | 2.04 | |
| | | Leu | 56 | HD13 | 2.18 | |
| | | Leu | 56 | HD22 | 3.44 | |
| | | Asp | 38 | HB1 | 3.33 | |
| | | Asp | 38 | O | 2.86 | |
| | HD2 | Tyr | 71 | CE2 | 3.14 | |
| | | Tyr | 71 | CZ | 3.58 | |
| | | Leu | 56 | HD12 | 3.31 | |
| | | Tyr | 71 | HE2 | 2.47 | |
| | | Tyr | 71 | OH | 3.37 | |
| | | Tyr | 71 | HH | 2.81 | |
| | | Asp | 38 | HB1 | 2.89 | |

TABLE 13

| | kobe2601 | H-RasT35S-GppNHp | | | | |
|---|---|---|---|---|---|---|
| model12 | atom | residue | number | atom | distance | angle |
| | C13 | Thr | 74 | HG22 | 3.24 | |
| | | Tyr | 71 | HE1 | 3.54 | |
| | C12 | Thr | 74 | HB | 3.38 | |
| | | Thr | 74 | HG22 | 3.41 | |
| | | Thr | 74 | HG1 | 3.25 | |
| | | Tyr | 71 | HE1 | 3.57 | |
| | | Tyr | 71 | HD1 | 3.43 | |
| | C11 | Tyr | 71 | HE1 | 3.09 | |
| | | Leu | 56 | HG | 3.48 | |
| | | Leu | 56 | HD21 | 3.17 | |
| | | Leu | 56 | HD22 | 3.53 | |
| | | Tyr | 71 | HD1 | 3.36 | |
| | F11 | Thr | 74 | HG1 | 3.52 | |
| | | Lys | 5 | CE | 3.11 | |
| | | Lys | 5 | HE2 | 3.12 | |
| | | Leu | 56 | HB2 | 3.34 | |
| | | Leu | 56 | CG | 3.28 | |
| | | Leu | 56 | HG | 2.67 | |
| | | Leu | 56 | CD2 | 3.04 | |
| | | Leu | 56 | HD21 | 2.86 | |
| | | Leu | 56 | HD22 | 2.64 | |
| | | Lys | 5 | HE1 | 2.54 | |
| | | Lys | 5 | NZ | 3.27 | |
| | | Lys | 5 | HZ3 | 2.56 | |
| | C10 | Tyr | 71 | CE1 | 3.45 | |
| | | Tyr | 71 | HE1 | 2.49 | |
| | | Leu | 56 | HD21 | 2.93 | |
| | | Leu | 56 | HD22 | 3.59 | |
| | C9 | Tyr | 71 | CE1 | 3.53 | |
| | | Tyr | 71 | HE1 | 2.45 | |
| | C8 | Tyr | 71 | HE1 | 3.03 | |
| | C7 | Gln | 70 | HG2 | 3.50 | |
| | S7 | Gln | 70 | HE22 | 3.34 | |
| | | Gln | 70 | CD | 3.37 | |
| | | Gln | 70 | NE2 | 3.00 | |
| | | Gln | 70 | HE21 | 2.88 | |
| | | Thr | 74 | HG22 | 3.01 | |
| | | Gln | 70 | HG2 | 3.27 | |
| | HAC | Gln | 70 | HE22 | 3.44 | |
| | | Gln | 70 | OE1 | 3.52 | |
| | C5 | Gln | 70 | OE1 | 3.34 | |
| | | Met | 67 | HB1 | 3.59 | |
| | | Gln | 70 | HG2 | 3.59 | |
| | C4 | Met | 67 | HA | 3.08 | |
| | | Met | 67 | CB | 3.36 | |
| | | Met | 67 | HB1 | 2.43 | |
| | | Met | 67 | HE1 | 3.30 | |
| | C3 | Met | 67 | HB1 | 2.58 | |
| | | Met | 67 | HE1 | 2.95 | |
| | | Met | 67 | HE3 | 3.17 | |
| | | Met | 67 | CE | 3.55 | |
| | N3 | Met | 67 | HN | 3.58 | |
| | | Met | 67 | CB | 3.33 | |
| | | Met | 67 | HG2 | 3.23 | |
| | | Met | 67 | HB1 | 2.37 | |
| | | Met | 67 | HE1 | 2.97 | |
| | | Met | 67 | HE3 | 2.56 | |
| | | Met | 67 | CE | 3.23 | |
| | O3B | Met | 67 | HB1 | 3.50 | |
| | | Met | 67 | HE1 | 3.51 | |
| | | Tyr | 64 | HE2 | 2.34 | |
| | | Tyr | 64 | CD2 | 3.26 | |
| | | Tyr | 64 | HD2 | 3.09 | |
| | | Tyr | 64 | CE2 | 2.89 | |
| | | Met | 67 | HE3 | 2.39 | |
| | | Met | 67 | CE | 3.37 | |
| | O3A | Ala | 66 | CB | 3.54 | |
| | | Ala | 66 | HB2 | 2.62 | |
| | | Ala | 66 | C | 3.48 | |
| | | Met | 67 | N | 2.54 | *** |
| | | Met | 67 | HN | 2.19 | |
| | | Met | 67 | CA | 2.85 | |
| | | Met | 67 | HA | 2.99 | |
| | | Met | 67 | CB | 2.57 | |
| | | Met | 67 | CG | 2.94 | |
| | | Met | 67 | HG2 | 2.37 | |
| | | Met | 67 | HB1 | 1.94 | |
| | | Met | 67 | HB2 | 3.56 | |
| | | Met | 67 | HE1 | 3.36 | |
| | | Tyr | 64 | HD2 | 3.27 | |
| | | Met | 67 | HE3 | 3.10 | |
| | C2 | Met | 67 | HE1 | 3.50 | |
| | H2 | Met | 67 | HE3 | 3.42 | |
| | H4 | Met | 67 | N | 3.32 | |
| | | Met | 67 | CA | 2.65 | |
| | | Met | 67 | HA | 2.00 | |
| | | Met | 67 | CB | 2.59 | |
| | | Met | 67 | HB1 | 1.88 | |
| | | Met | 67 | HB2 | 3.02 | |
| | | Met | 67 | HE1 | 3.34 | |
| | | Gln | 70 | HB1 | 3.27 | |
| | H5 | Gln | 70 | CG | 3.23 | |
| | | Gln | 70 | CD | 3.05 | |
| | | Gln | 70 | OE1 | 2.78 | |
| | | Gln | 70 | HG2 | 2.57 | |
| | HD1 | Tyr | 71 | HE1 | 2.76 | |
| | | Glu | 37 | HG1 | 3.44 | |
| | HE1 | Tyr | 71 | CE1 | 3.45 | |
| | | Tyr | 71 | HE1 | 2.62 | |
| | | Leu | 56 | CD2 | 3.01 | |
| | | Leu | 56 | HD21 | 2.23 | |
| | | Leu | 56 | HD22 | 2.91 | |
| | | Glu | 37 | HG1 | 3.23 | |
| | HE2 | Thr | 74 | CB | 2.96 | |
| | | Thr | 74 | CG2 | 3.17 | |
| | | Thr | 74 | HG23 | 3.20 | |
| | | Thr | 74 | HB | 2.34 | |
| | | Thr | 74 | HG22 | 2.84 | |
| | | Thr | 74 | OG1 | 3.05 | |
| | | Thr | 74 | HG1 | 2.46 | |
| | | Lys | 5 | CE | 3.59 | |
| | | Lys | 5 | HE2 | 2.78 | |
| | | Tyr | 71 | HD1 | 3.53 | |
| | | Lys | 5 | HE1 | 3.57 | |
| | HD2 | Gln | 70 | HE21 | 3.60 | |
| | | Thr | 74 | CG2 | 3.21 | |
| | | Thr | 74 | HG23 | 3.10 | |
| | | Thr | 74 | HG22 | 2.44 | |
| | | Gln | 70 | HG2 | 3.17 | |

TABLE 14

| | kobe2601 | H-RasT35S-GppNHp | | | | |
|---|---|---|---|---|---|---|
| model13 | atom | residue | number | atom | distance | angle |
| | C13 | Gln | 70 | HG1 | 2.92 | |
| | | Gln | 70 | HG2 | 3.33 | |
| | | Thr | 74 | HG23 | 3.34 | |
| | | Tyr | 71 | CD2 | 3.11 | |
| | | Tyr | 71 | HD2 | 2.96 | |
| | | Tyr | 71 | CE2 | 2.90 | |
| | | Tyr | 71 | HE2 | 2.54 | |
| | C12 | Gln | 70 | HG1 | 3.57 | |
| | | Thr | 74 | HG23 | 3.21 | |
| | | Tyr | 71 | CD2 | 3.21 | |
| | | Tyr | 71 | HD2 | 2.65 | |
| | | Tyr | 71 | CE2 | 3.20 | |
| | | Tyr | 71 | HE2 | 2.63 | |
| | | Thr | 74 | OG1 | 3.37 | |
| | C11 | Tyr | 71 | HE2 | 3.52 | |
| | | Lys | 5 | HE1 | 3.59 | |
| | F11 | Lys | 5 | CE | 3.27 | |
| | | Lys | 5 | HE1 | 2.56 | |
| | | Lys | 5 | NZ | 3.17 | |
| | | Lys | 5 | HZ2 | 3.29 | |
| | | Lys | 5 | HZ3 | 2.57 | |
| | C8 | Gln | 70 | HG1 | 3.43 | |
| | | Gln | 70 | HG2 | 3.11 | |
| | | Tyr | 71 | HE2 | 3.39 | |
| | N8 | Gln | 70 | HG2 | 2.80 | |
| | HAD | Gln | 70 | HG2 | 3.25 | |
| | C7 | Gln | 70 | HB1 | 2.94 | |
| | | Gln | 70 | CG | 3.42 | |
| | | Gln | 70 | HG1 | 3.48 | |
| | | Gln | 70 | HG2 | 2.72 | |
| | | Met | 67 | HA | 2.82 | |
| | | Met | 67 | HG1 | 2.80 | |
| | | Met | 67 | HB1 | 3.38 | |
| | S7 | Met | 67 | C | 3.32 | |
| | | Met | 67 | O | 2.64 | |
| | | Gln | 70 | CB | 3.26 | |
| | | Gln | 70 | HB1 | 2.42 | |
| | | Gln | 70 | CG | 3.30 | |
| | | Gln | 70 | HG1 | 2.97 | |
| | | Gln | 70 | HG2 | 3.14 | |
| | | Tyr | 71 | HN | 3.22 | |
| | | Tyr | 71 | N | 3.50 | |
| | | Tyr | 71 | CG | 3.29 | |
| | | Tyr | 71 | CD1 | 3.43 | |
| | | Tyr | 71 | CD2 | 3.00 | |
| | | Tyr | 71 | HD2 | 3.51 | |
| | | Tyr | 71 | CE1 | 3.34 | |
| | | Tyr | 71 | CE2 | 2.90 | |
| | | Tyr | 71 | HE2 | 3.35 | |
| | | Met | 67 | CA | 3.35 | |
| | | Met | 67 | HA | 2.81 | |
| | | Met | 67 | CB | 3.57 | |
| | | Met | 67 | HB1 | 2.94 | |
| | | Tyr | 71 | CZ | 3.08 | |
| | N7 | Gln | 70 | HB1 | 3.16 | |
| | | Gln | 70 | HG2 | 3.31 | |
| | | Met | 67 | CA | 3.06 | |
| | | Met | 67 | HA | 2.18 | |
| | | Met | 67 | CB | 3.19 | |
| | | Met | 67 | CG | 2.82 | |
| | | Met | 67 | HG1 | 1.86 | |
| | | Met | 67 | HG2 | 3.54 | |
| | | Met | 67 | CE | 3.35 | |
| | | Met | 67 | HE2 | 3.08 | |
| | | Met | 67 | HE3 | 2.89 | |
| | | Met | 67 | HB1 | 3.22 | |
| | HAC | Ala | 66 | O | 3.29 | |
| | | Met | 67 | C | 3.28 | |
| | | Met | 67 | O | 3.52 | |
| | | Gln | 70 | HB1 | 2.95 | |
| | | Ala | 66 | C | 3.42 | |
| | | Met | 67 | N | 2.95 | |
| | | Met | 67 | HB2 | 3.47 | |
| | | Met | 67 | CA | 2.11 | |
| | | Met | 67 | HA | 1.26 | |
| | | Met | 67 | CB | 2.44 | |
| | | Met | 67 | CG | 2.32 | |
| | | Met | 67 | HG1 | 1.64 | |
| | | Met | 67 | HG2 | 3.24 | |
| | | Met | 67 | SD | 3.27 | |
| | | Met | 67 | CE | 2.98 | |
| | | Met | 67 | HE2 | 2.57 | |
| | | Met | 67 | HE3 | 2.84 | |
| | | Met | 67 | HB1 | 2.71 | |
| | N6 | Met | 67 | HA | 3.16 | |
| | | Met | 67 | CG | 3.21 | |
| | | Met | 67 | HG1 | 2.17 | |
| | | Met | 67 | CE | 2.95 | |
| | | Met | 67 | HE2 | 2.88 | |
| | | Met | 67 | HE3 | 2.19 | |
| | HAB | Met | 67 | CG | 2.93 | |
| | | Met | 67 | HG1 | 1.96 | |
| | | Met | 67 | HG2 | 3.34 | |
| | | Met | 67 | SD | 3.52 | |
| | | Met | 67 | CE | 2.71 | |
| | | Met | 67 | HE1 | 3.50 | |
| | | Met | 67 | HE2 | 3.01 | |
| | | Met | 67 | HE3 | 1.74 | |
| | C6 | Gln | 70 | HG2 | 3.50 | |
| | | Met | 67 | HG1 | 3.46 | |
| | | Met | 67 | CE | 3.54 | |
| | | Met | 67 | HE2 | 3.29 | |
| | | Met | 67 | HE3 | 2.83 | |
| | C5 | Gln | 70 | HB1 | 3.57 | |
| | | Gln | 70 | HB2 | 3.25 | |
| | | Gln | 70 | CG | 3.34 | |
| | | Gln | 70 | HG2 | 2.43 | |
| | C4 | Gln | 70 | HG2 | 3.01 | |
| | C1 | Met | 67 | HE3 | 3.08 | |
| | N1 | Met | 67 | HE3 | 2.77 | |
| | O1A | Met | 67 | HG1 | 3.27 | |
| | | Met | 67 | CE | 3.12 | |
| | | Met | 67 | HE1 | 3.48 | |
| | | Met | 67 | HE3 | 2.06 | |
| | H4 | Gln | 70 | HB2 | 3.38 | |
| | | Gln | 70 | CG | 3.37 | |
| | | Gln | 70 | HG2 | 2.81 | |
| | | Gln | 70 | CD | 3.10 | |
| | | Gln | 70 | OE1 | 3.56 | |
| | | Gln | 70 | NE2 | 3.19 | |
| | | Gln | 70 | HE21 | 3.34 | |
| | | Gln | 70 | HE22 | 3.56 | |
| | H5 | Gln | 70 | CB | 2.59 | |
| | | Gln | 70 | HB1 | 2.52 | |
| | | Gln | 70 | HB2 | 2.42 | |
| | | Gln | 70 | CG | 2.41 | |
| | | Gln | 70 | HG1 | 3.21 | |
| | | Gln | 70 | HG2 | 1.59 | |
| | | Gln | 70 | CD | 3.33 | |
| | | Met | 67 | HA | 3.31 | |
| | HE2 | Thr | 74 | HG23 | 3.46 | |
| | | Leu | 56 | HD22 | 3.09 | |
| | | Tyr | 71 | HA | 3.01 | |
| | | Tyr | 71 | CD2 | 2.61 | |
| | | Tyr | 71 | HD2 | 1.81 | |
| | | Tyr | 71 | CE2 | 2.94 | |
| | | Tyr | 71 | HE2 | 2.55 | |
| | | Thr | 74 | OG1 | 2.83 | |
| | | Thr | 74 | HG1 | 3.02 | |
| | | Lys | 5 | HE1 | 3.21 | |
| | HD2 | Gln | 70 | HB1 | 3.25 | |
| | | Gln | 70 | CG | 3.12 | |
| | | Gln | 70 | HG1 | 2.40 | |
| | | Gln | 70 | HG2 | 3.04 | |
| | | Thr | 74 | HG23 | 3.34 | |
| | | Tyr | 71 | N | 3.58 | |
| | | Tyr | 71 | HA | 3.17 | |
| | | Tyr | 71 | CG | 3.40 | |
| | | Tyr | 71 | CD2 | 2.59 | |
| | | Tyr | 71 | HD2 | 2.63 | |
| | | Tyr | 71 | CE2 | 2.65 | |
| | | Tyr | 71 | HE2 | 2.73 | |
| | | Tyr | 71 | CZ | 3.48 | |

TABLE 15

| | kobe2601 | H-RasT35S-GppNHp | | | | |
|---|---|---|---|---|---|---|
| model14 | atom | residue | number | atom | distance | angle |
| | C13 | Gln | 70 | HE22 | 3.31 | |
| | C11 | Thr | 74 | HG21 | 3.34 | |
| | | Thr | 74 | HB | 3.48 | |
| | | Lys | 5 | HE2 | 3.44 | |
| | | Lys | 5 | HZ3 | 3.28 | |
| | F11 | Thr | 74 | HB | 3.57 | |
| | | Lys | 5 | CE | 3.33 | |
| | | Lys | 5 | HE1 | 3.58 | |
| | | Lys | 5 | HE2 | 2.54 | |
| | | Lys | 5 | NZ | 3.52 | |
| | | Lys | 5 | HZ3 | 2.88 | |
| | C10 | Thr | 74 | HG21 | 2.69 | |
| | | Thr | 74 | HG1 | 3.15 | |
| | | Thr | 74 | HB | 3.19 | |
| | | Gln | 70 | OE1 | 3.30 | |
| | | Gln | 70 | HE22 | 3.59 | |
| | | Lys | 5 | HE1 | 3.34 | |
| | | Lys | 5 | HE2 | 3.54 | |
| | | Lys | 5 | HZ3 | 2.95 | |
| | C9 | Thr | 74 | CG2 | 3.55 | |
| | | Thr | 74 | HG21 | 2.49 | |
| | | Thr | 74 | HG1 | 3.43 | |
| | | Gln | 70 | CD | 3.10 | |
| | | Gln | 70 | OE1 | 2.63 | |
| | | Gln | 70 | NE2 | 3.10 | |
| | | Gln | 70 | HE22 | 2.64 | |
| | C8 | Thr | 74 | HG21 | 3.01 | |
| | | Gln | 70 | CD | 3.56 | |
| | | Gln | 70 | OE1 | 3.53 | |
| | | Gln | 70 | NE2 | 3.03 | |
| | | Gln | 70 | HE22 | 2.45 | |
| | N8 | Gln | 70 | CD | 3.31 | |
| | | Gln | 70 | NE2 | 2.62 | *** |
| | | Gln | 70 | CG | 3.49 | |
| | | Gln | 70 | HG2 | 2.69 | |
| | | Gln | 70 | HE21 | 3.22 | |
| | HAC | Gln | 70 | CD | 2.57 | |
| | | Gln | 70 | OE1 | 3.27 | |
| | | Gln | 70 | NE2 | 2.35 | |
| | | Gln | 70 | HE22 | 2.77 | |
| | | Gln | 70 | CG | 2.98 | |
| | | Gln | 70 | HG1 | 3.53 | |
| | | Gln | 70 | HG2 | 2.45 | |
| | | Gln | 70 | HE21 | 2.40 | |
| | C6 | Met | 67 | HE1 | 3.29 | |
| | C5 | Gln | 70 | HG2 | 3.34 | |
| | | Met | 67 | HE1 | 3.32 | |
| | C4 | Met | 67 | HB1 | 3.02 | |
| | | Met | 67 | CE | 3.55 | |
| | | Met | 67 | HE1 | 2.95 | |
| | | Met | 67 | HE2 | 3.28 | |
| | C3 | Met | 67 | CE | 2.93 | |
| | | Met | 67 | HE1 | 2.47 | |
| | | Met | 67 | HG1 | 3.60 | |
| | | Met | 67 | HE2 | 2.47 | |
| | N3 | Met | 67 | CE | 3.05 | |
| | | Met | 67 | HE1 | 3.06 | |
| | | Met | 67 | HG1 | 3.02 | |
| | | Met | 67 | HE2 | 2.18 | |
| | | Tyr | 64 | HH | 3.37 | |
| | O3B | Met | 67 | HB1 | 3.16 | |
| | | Met | 67 | CG | 3.54 | |
| | | Met | 67 | HG1 | 2.62 | |
| | | Met | 67 | HE2 | 2.84 | |
| | | Tyr | 71 | OH | 2.72 | *** |
| | | Tyr | 71 | HH | 3.20 | |
| | O3A | Met | 67 | CE | 3.33 | |
| | | Met | 67 | HE1 | 3.55 | |
| | | Tyr | 64 | CZ | 3.12 | |
| | | Tyr | 64 | OH | 2.59 | *** |
| | | Met | 67 | HE2 | 2.42 | |
| | | Tyr | 64 | HH | 2.28 | |
| | C2 | Met | 67 | CE | 3.09 | |
| | | Met | 67 | HE1 | 2.42 | |
| | | Met | 67 | HE3 | 3.52 | |
| | | Met | 67 | HE2 | 2.94 | |
| | C1 | Met | 67 | HE1 | 2.86 | |
| | H2 | Met | 67 | CE | 3.24 | |
| | | Met | 67 | HE1 | 2.80 | |
| | | Met | 67 | HE3 | 3.36 | |
| | | Met | 67 | HE2 | 3.02 | |
| | H4 | Met | 67 | CB | 3.55 | |
| | | Met | 67 | HB1 | 2.50 | |
| | | Tyr | 71 | CE2 | 3.21 | |
| | | Tyr | 71 | OH | 2.99 | |
| | | Tyr | 71 | HE2 | 2.85 | |
| | | Tyr | 71 | CZ | 3.29 | |
| | H5 | Gln | 70 | HB1 | 3.41 | |
| | | Gln | 70 | CG | 3.45 | |
| | | Gln | 70 | HG2 | 2.41 | |
| | HE1 | Glu | 37 | HB2 | 3.33 | |
| | | Glu | 37 | HG1 | 3.23 | |
| | | Glu | 37 | CD | 3.26 | |
| | | Glu | 37 | OE1 | 3.02 | |
| | HE2 | Thr | 74 | CB | 3.52 | |
| | | Thr | 74 | HG21 | 3.52 | |
| | | Thr | 74 | HG23 | 3.36 | |
| | | Thr | 74 | HG1 | 3.02 | |
| | | Lys | 5 | CE | 3.49 | |
| | | Lys | 5 | HE1 | 3.07 | |
| | | Lys | 5 | NZ | 2.84 | |
| | | Lys | 5 | HZ2 | 2.60 | |
| | | Lys | 5 | HZ3 | 2.30 | |
| | | Thr | 74 | HB | 2.79 | |
| | HD2 | Gln | 70 | HG2 | 3.54 | |
| | | Thr | 74 | CG2 | 2.88 | |
| | | Thr | 74 | HG21 | 2.44 | |
| | | Thr | 74 | HG23 | 2.43 | |
| | | Thr | 74 | HB | 3.59 | |

TABLE 16

| | kobe2601 | H-RasT35S-GppNHp | | | | |
|---|---|---|---|---|---|---|
| model15 | atom | residue | number | atom | distance | angle |
| | C13 | Thr | 74 | HG21 | 3.38 | |
| | | Thr | 74 | HG23 | 3.35 | |
| | C12 | Lys | 5 | NZ | 3.59 | |
| | | Lys | 5 | HZ2 | 3.33 | |
| | | Lys | 5 | HZ3 | 2.92 | |
| | | Glu | 37 | OE1 | 3.39 | |
| | C11 | Lys | 5 | HZ2 | 3.37 | |
| | | Lys | 5 | HZ3 | 3.10 | |
| | | Glu | 37 | CD | 3.54 | |
| | | Glu | 37 | OE1 | 2.85 | |
| | F11 | Lys | 5 | NZ | 2.94 | |
| | | Lys | 5 | HZ1 | 3.17 | |
| | | Lys | 5 | HZ2 | 2.58 | |
| | | Lys | 5 | HZ3 | 2.57 | |
| | | Glu | 37 | CD | 3.44 | |
| | | Glu | 37 | OE1 | 3.16 | |
| | | Glu | 37 | OE2 | 3.33 | |
| | C10 | Glu | 37 | CD | 3.55 | |
| | | Glu | 37 | OE1 | 2.94 | |
| | C9 | Glu | 37 | OE1 | 3.53 | |
| | C7 | Gln | 70 | HG2 | 2.68 | |
| | | Gln | 70 | HE21 | 3.49 | |
| | S7 | Gln | 70 | NE2 | 3.52 | |
| | | Gln | 70 | CG | 3.13 | |
| | | Gln | 70 | HG1 | 2.92 | |
| | | Gln | 70 | HG2 | 2.49 | |
| | | Gln | 70 | HE21 | 2.80 | |
| | | Thr | 74 | CG2 | 2.97 | |
| | | Thr | 74 | HG21 | 2.64 | |
| | | Thr | 74 | HG22 | 3.40 | |
| | | Thr | 74 | HG23 | 2.47 | |
| | N7 | Gln | 70 | CD | 3.41 | |
| | | Gln | 70 | NE2 | 3.31 | * |
| | | Gln | 70 | CG | 3.49 | |
| | | Gln | 70 | HG2 | 2.69 | |
| | | Gln | 70 | HE21 | 3.22 | |
| | HAC | Gln | 70 | CD | 2.57 | |
| | | Gln | 70 | OE1 | 3.27 | |
| | | Gln | 70 | NE2 | 2.35 | |
| | | Gln | 70 | HE22 | 2.77 | |
| | | Gln | 70 | CG | 2.98 | |
| | | Gln | 70 | HG1 | 3.53 | |
| | | Gln | 70 | HG2 | 2.45 | |
| | | Gln | 70 | HE21 | 2.40 | |
| | C6 | Met | 67 | HE1 | 3.29 | |
| | C5 | Gln | 70 | HG2 | 3.34 | |
| | | Met | 67 | HE1 | 3.32 | |
| | C4 | Met | 67 | HB1 | 3.02 | |
| | | Met | 67 | CE | 3.55 | |
| | | Met | 67 | HE1 | 2.95 | |
| | | Met | 67 | HE2 | 3.28 | |
| | C3 | Met | 67 | CE | 2.93 | |
| | | Met | 67 | HE1 | 2.47 | |
| | | Met | 67 | HG1 | 3.60 | |
| | | Met | 67 | HE2 | 2.47 | |
| | N3 | Met | 67 | CE | 3.05 | |
| | | Met | 67 | HE1 | 3.06 | |
| | | Met | 67 | HG1 | 3.02 | |
| | | Met | 67 | HE2 | 2.18 | |
| | | Tyr | 64 | HH | 3.37 | |
| | O3B | Met | 67 | HB1 | 3.16 | |
| | | Met | 67 | CG | 3.54 | |
| | | Met | 67 | HG1 | 2.62 | |
| | | Met | 67 | HE2 | 2.84 | |
| | | Tyr | 71 | OH | 2.72 | *** |
| | | Tyr | 71 | HH | 3.20 | |
| | O3A | Met | 67 | CE | 3.33 | |
| | | Met | 67 | HE1 | 3.55 | |
| | | Tyr | 64 | CZ | 3.12 | |
| | | Tyr | 64 | OH | 2.59 | *** |
| | | Met | 67 | HE2 | 2.42 | |
| | | Tyr | 64 | HH | 2.28 | |
| | C2 | Met | 67 | CE | 3.09 | |
| | | Met | 67 | HE1 | 2.42 | |
| | | Met | 67 | HE3 | 3.52 | |
| | | Met | 67 | HE2 | 2.94 | |
| | C1 | Met | 67 | HE1 | 2.86 | |
| | H2 | Met | 67 | CE | 3.24 | |
| | | Met | 67 | HE1 | 2.80 | |
| | | Met | 67 | HE3 | 3.36 | |
| | | Met | 67 | HE2 | 3.02 | |
| | H4 | Met | 67 | CB | 3.55 | |
| | | Met | 67 | HB1 | 2.50 | |
| | | Tyr | 71 | CE2 | 3.21 | |
| | | Tyr | 71 | OH | 2.99 | |
| | | Tyr | 71 | HE2 | 2.85 | |
| | | Tyr | 71 | CZ | 3.29 | |
| | H5 | Gln | 70 | HB1 | 3.41 | |
| | | Gln | 70 | CG | 3.45 | |
| | | Gln | 70 | HG2 | 2.41 | |
| | HE1 | Glu | 37 | HB2 | 3.33 | |
| | | Glu | 37 | HG1 | 3.23 | |
| | | Glu | 37 | CD | 3.26 | |
| | | Glu | 37 | OE1 | 3.02 | |
| | HE2 | Thr | 74 | CB | 3.52 | |
| | | Thr | 74 | HG21 | 3.52 | |
| | | Thr | 74 | HG23 | 3.36 | |
| | | Thr | 74 | HG1 | 3.02 | |
| | | Lys | 5 | CE | 3.49 | |
| | | Lys | 5 | HE1 | 3.07 | |
| | | Lys | 5 | NZ | 2.84 | |
| | | Lys | 5 | HZ2 | 2.60 | |
| | | Lys | 5 | HZ3 | 2.30 | |
| | | Thr | 74 | HB | 2.79 | |
| | HD2 | Gln | 70 | HG2 | 3.54 | |
| | | Thr | 74 | CG2 | 2.88 | |
| | | Thr | 74 | HG21 | 2.44 | |
| | | Thr | 74 | HG23 | 2.43 | |
| | | Thr | 74 | HB | 3.59 | |

### INDUSTRIAL APPLICABILITY

As described above, it was possible to confirm the amino acid residues at the site important for the interaction between the Ras polypeptide and the Ras inhibitor. Screening can be effectively carried out by screening for a substance capable of interacting with the important amino acid residues. In addition, the structure of the Ras inhibitor (A), which is a derivative of the Ras inhibitor candidate (a), can be efficiently estimated by confirming the difference between the structural information on the Ras polypeptide by NMR and the structural information on the complex between the Ras polypeptide and the seed compound by NMR. That is, a novel derivative can be designed and synthesized in such a form that a substituent at a high risk of expressing toxicity or a metabolically unstable chemical structure is substituted by its biological equivalent in the structure of a structurally modified compound obtained by modifying the Ras inhibitor candidate (a), and the Ras inhibitor (A) that can serve as a lead compound can be derived.

The resultant Ras inhibitor (A) is considered to have activities of treating and preventing various diseases that may be developed based on the aberrant Ras function, such as an anti-cancer activity. Thus, the Ras inhibitor (A) in the present invention may be used for drugs each containing the Ras inhibitor as an active ingredient, such as therapeutic drugs for tumors including anti-cancer drugs.

1-12. (canceled)

13. A screening method for identifying a Ras inhibitor (A), comprising the steps of:
obtaining an NMR spectrum from a partial Ras polypeptide of an amino acid sequence set forth in SEQ ID NO: 1 or an amino acid sequence having substitutions, deletions, or additions of one to three amino acids in the amino acid sequence set forth in SEQ ID NO: 1;
(ii) obtaining an NMR spectrum of a complex produced by contacting a Ras inhibitor candidate with said partial Ras polypeptide; and
(iii) identifying whether said Ras inhibitor candidate (a) is a Ras inhibitor (A) by analyzing a difference between the NMR spectrum obtained in said step (i) and the NMR spectrum obtained in said step (ii).

14. The screening method for identifying a Ras inhibitor (A) according to claim 13, wherein said partial Ras polypeptide comprises a polypeptide in which threonine at position 35 is substituted by serine in the amino acid sequence set forth in SEQ ID NO: 1.

15. The screening method for identifying a Ras inhibitor (A) according to claim 13, wherein said Ras inhibitor candidate (a) is of the formula (I): 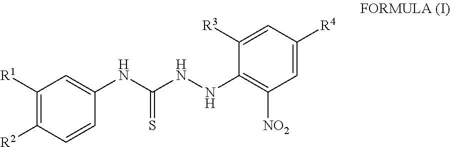
wherein each of R¹, R², R³, and R⁴ is independently selected from the group consisting of hydrogen, halogen, a lower alkyl group, a nitro group, and a trifluoromethyl group.

16. A screening method for identifying a Ras inhibitor candidate (a), comprising:
determining whether a substance interacts with at least three or more reference amino acid residues, wherein said reference amino acid residues are selected from the group consisting of, with reference to an amino acid sequence set forth in SEQ ID NO: 1, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74 in a partial Ras polypeptide of the amino acid sequence set forth in SEQ ID NO: 1 or an amino acid sequence having substitutions, deletions, or additions of one to three amino acids in the amino acid sequence set forth in SEQ ID NO: 1; and
identifying a substance that interacts with at least three or more reference amino acid residues as a Ras inhibitor candidate (a).

17. The screening method for identifying a Ras inhibitor candidate (a) according to claim 16 further comprising the steps of:
(i) producing a polypeptide in which at least three or more said reference amino acid residues are labeled with an isotope measurable by NMR to produce a labeled polypeptide;
(ii) obtaining an NMR spectrum of said labeled polypeptide of said step (i);
(iii) contacting a candidate with said labeled polypeptide to form a complex;
(iv) obtaining an NMR spectrum of said complex; and
(iii) identifying, as the Ras inhibitor candidate (a), a substance that interacts with said labeled polypeptide based on a difference between the NMR spectrum of said labeled polypeptide and the NMR spectrum of said complex.

18. The screening method for identifying a Ras inhibitor candidate (a) according to claim 16, wherein said partial Ras polypeptide comprises a polypeptide in which threonine at position 35 is substituted by serine in the amino acid sequence set forth in SEQ ID NO: 1.

19. A Ras inhibitor of the formula (I): 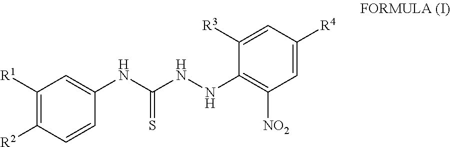
wherein each of R¹, R², R³, and R⁴ is independently selected from the group consisting of hydrogen, halogen, a lower alkyl group, a nitro group, and a trifluoromethyl group.

20. A complex between a partial Ras polypeptide of an amino acid sequence set forth in SEQ ID NO: 1 or an amino acid sequence having substitutions, deletions, or additions of one to three amino acids in the amino acid sequence set forth in SEQ ID NO: 1, and a Ras inhibitor candidate (a) of the formula (I): 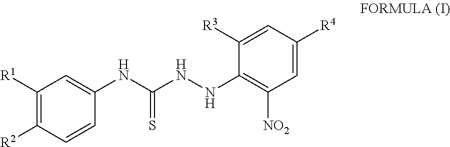
wherein each of R¹, R², R³, and R⁴ is independently selected from the group consisting of hydrogen, halogen, a lower alkyl group, a nitro group, and a trifluoromethyl group.

21. The complex according to claim 20, wherein said partial Ras polypeptide comprises a polypeptide in which threonine at position 35 is substituted by serine in the amino acid sequence set forth in SEQ ID NO: 1.

22. The complex according to claim 20, wherein said complex is specified by an NMR signal.

23. An aqueous solution comprising a partial Ras polypeptide of an amino acid sequence set forth in SEQ ID NO: 1 or an amino acid sequence having substitutions, deletions, or additions of one to three amino acids in the amino acid sequence set forth in SEQ ID NO: 1, wherein the structure of said partial Ras polypeptide comprises a conformation having a pocket that binds to a specified substance, and wherein said specified substance comprises a substance that interacts with at least three or more reference amino acid residues, wherein said reference amino acid residues are selected from the group consisting of, with reference to the amino acid sequence set forth in SEQ ID NO: 1, lysine (K) at position 5, glutamic acid (E) at position 37, aspartic acid (D) at position 38, serine (S) at position 39, leucine (L) at position 56, glutamic acid (E) at position 63, tyrosine (Y) at position 64, alanine (A) at position 66, methionine (M) at position 67, glutamine (Q) at position 70, tyrosine (Y) at position 71, arginine (R) at position 73, and threonine (T) at position 74.

24. The aqueous solution according to claim 23, wherein said partial Ras polypeptide comprises a polypeptide in which threonine at position 35 is substituted by serine in the amino acid sequence set forth in SEQ ID NO: 1.

25. The aqueous solution according to claim 23, wherein the specified substance comprises a compound of the formula: 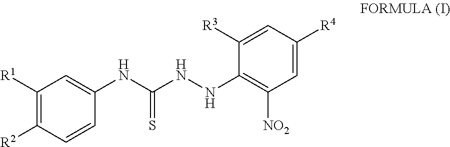
wherein each of R¹, R², R³, and R⁴ is independently selected from the group consisting of hydrogen, halogen, a lower alkyl group, a nitro group, and a trifluoromethyl group.


SEQUENCE LISTING (1 sequence)

SEQ ID NO: 1 - protein, 166 aa, Homo sapiens [sequence omitted]